(19) United States
(12) Patent Application Publication
(10) Pub. No.: US 2026/0091103 A1
(43) Pub. Date: Apr. 2, 2026

(54) NOVEL COMPOSITIONS COMPRISING TERPENOID AND MACROMOLECULE, AND USES THEREOF

(71) Applicants: CHIH-AN LIN, Dongguan City, Guangdong (CN); Powin Biomedical Co., Ltd., Taichung City (TW)

(72) Inventors: CHIH-AN LIN, Dongguan City (TW); PO-CHENG CHU, Taoyuan City (TW); MIN-HAN ZHAO, Yilan City (TW); CHAN-CHANG HU, New Taipei City (TW); TZUNG-JING MING, Taipei City (TW); GUO-DONG WU, Jinan City (CN)

(73) Assignees: CHIH-AN LIN, Dongguan City, Guangdong (CN); Powin Biomedical Co., Ltd., Taichung City (TW)

(21) Appl. No.: 19/115,011

(22) PCT Filed: Sep. 26, 2022
(86) PCT No.: PCT/CN2022/121455

§ 371 Date: Mar. 25, 2025

(51) Int. Cl. A61K 39/145; A61K 39/00; A61P 37/04

(52) U.S. Cl. CPC A61K 39/145; A61K 39/0011; A61P 37/04; A61K 2039/6018; A61K 2039/6087; A61K 2039/804

(57) ABSTRACT

Provided herein are novel compositions comprising oil@HA-derivative particles with high potential for the use in the stimulation of immune response. Methods to produce such compositions and methods of uses thereof in treating or preventing a disease or disorder are also provided.

### FIELD OF THE INVENTION

The present invention relates to a composition comprising oil such as terpenoid and macromolecule, and the uses thereof, specifically, relates to an oil-in-water composition comprising oil such as terpenoid and macromolecule, and the uses thereof.

### BACKGROUND OF THE INVENTION

Vaccination is the most effective strategy to prevent or limit the severity of diseases such as infection-associated syndromes and cancer. For safety issues, new-generation vaccine candidates usually employ a highly purified sub-portion of the target cell (such as sub-portion of the target pathogen or sub-portion of the target tumor cell) as an antigen; however, these components often lack immunogenicity, thus necessitate adjuvants to facilitate the induction of adaptive immunity. Currently, the most common salts for large-scale vaccination are aluminum-based mineral salts that mainly function as a depot to drive humoral immunity; however, they are usually limited by the weak stimulation of cell-mediated immunity.

Several developmental adjuvants have been approved or authorized in prophylactic human vaccines for enhanced cell-mediated immunity, and the example includes MF59. Nevertheless, squalene-based adjuvants like MF59 were unsuitable for cancer vaccine use due to the poor cytotoxic T-lymphocytes (CTL) response. Thus, there is an unmet need to develop an efficient adjuvant which could stimulate immune responses such as T-lymphocytes responses.

### SUMMARY OF THE INVENTION

Accordingly, it is an object of the present invention to provide an efficient adjuvant suitable for stimulating immune responses in host animals such as in mammals.

It is another object of the present invention to provide an efficient adjuvant suitable for drive humoral immunity and/or cell-mediated immunity.

It is a further object of the present invention to provide an useful vaccine composition.

It is a further object of the present invention to provide an efficient adjuvant suitable for cancer vaccine use.

It is a further object of the present invention to provide a method of stimulating an immune response or treating cancer, tumor, infection, and other diseases in a subject.

Provided herein are compositions comprising oil and macromolecule; the oil and macromolecule together form a plurality of particles; the macromolecule comprises:
at least one first unit, wherein the first unit is a first disaccharide; and
at least one second unit, wherein the second unit is a conjugation of a second disaccharide and a hydrophobic compound;
wherein the at least one first unit and the at least one second unit are linked to each other to form the macromolecule.

In some embodiments, the oil is a terpenoid.

In some embodiments, the first disaccharide is composed of D-glucuronic acid and N-acetylglucosamine.

In some embodiments, the second disaccharide is composed of D-glucuronic acid and N-acetylglucosamine; and in the second unit, the hydrophobic compound is attached to carbon 6 of the D-glucuronic acid.

In some embodiments, the D-glucuronic acid and N-acetylglucosamine in the first disaccharide are linked by a β-(1,3) bond.

In some embodiments, the N-acetylglucosamine and the D-glucuronic acid in the second disaccharide are linked by a β-(1,3) bond.

In some embodiments, the at least one first unit and the at least one second unit are linked to each other by a β-(1,4) bond to form the macromolecule.

In some embodiments, the hydrophobic compound is selected from the group consisting of cholesterol derivatives, octadecylamine, octadecylamine derivatives, steroids, steroid derivatives, saturated or unsaturated long chain fatty amines, and saturated or unsaturated long chain fatty amine derivatives, or any combination thereof. As used herein, the term “derivatives” refers to products obtained through chemical reactions, to modify the original compound (such as, cholesterol, octadecylamine, steroids, saturated or unsaturated long chain fatty amines) into derivatives with primary amine groups. In some embodiments, examples of cholesterol derivative include, but not limited to, cholesterol-derived amines, cholesterol-amino acid conjugates, or any combination thereof. In some embodiments, the amino acid in the cholesterol-amino acid conjugates is selected from the group consisting of amino acids whose main carbon chain has two to six carbon atoms, such as glycine, alanine, valine, leucine, isoleucine, etc.

In some embodiments, the oil is selected from the group consisting of squalene, squalane, ocimene, famesene, and paraffin oil, or any combination thereof.

In some embodiments, in the composition, the weight ratio of the oil and the macromolecule ranges from 1:100 to 80:1.

In some embodiments, the weight percentage of the oil is equal to or more than 0.01% to 40%, based on the total weight of the composition.

In some embodiments, the weight percentage of the macromolecule is equal to or more than 0.001% to 3%, based on the total weight of the composition.

In some embodiments, at least 50% (w/w) of oil in the composition is encapsulated in the particles, based on the total content of oil in the composition being 100% by mass.

In some embodiments, the composition comprises at least 1% (v/v) of oil, based on the total volume of the composition.

In some embodiments, the macromolecule is prepared by a method comprising mixing 0.0001˜2 equivalent of the hydrophobic compound with 1 equivalent of hyaluronic acid.

In some embodiments, in the composition, the molar ratio of the first unit and the second unit per macromolecule on average ranges from 99:1 to 1:1.

Provided herein are also compositions comprising terpenoid and macromolecule; the terpenoid and macromolecule together form a plurality of particles; the macromolecule comprises:
at least one first unit, wherein the first unit is a disaccharide in which D-glucuronic acid is linked to N-acetylglucosamine; and
at least one second unit, wherein the second unit is a disaccharide-derivative in which D-glucuronic acid is linked to N-acetylglucosamine and a hydrophobic compound is attached to carbon 6 of said D-glucuronic acid;
wherein the at least one first unit and the at least one second unit are linked to each other to form the macromolecule.

In some embodiments, the D-glucuronic acid and N-acetylglucosamine in the disaccharide are linked by a β-(1,3) bond.

In some embodiments, the N-acetylglucosamine and the D-glucuronic acid attached by the hydrophobic compound in the disaccharide-derivative are linked by a β-(1,3) bond.

In some embodiments, the at least one first unit and the at least one second unit are linked to each other by a β-(1,4) bond to form the macromolecule.

In some embodiments, the macromolecule is a polymer.

In some embodiments, the macromolecule is a hyaluronic acid derivative.

In some embodiments, the macromolecule is a hyaluronic acid-hydrophobic compound conjugate.

In some embodiments, the macromolecule is a compound of formula (I):

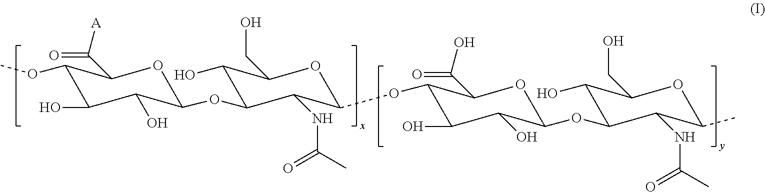

And wherein A group is R—NH— or R—X—NH—, wherein R group is a hydrophobic group derived from the hydrophobic compound, X denotes a hetero-atom (such as O, S, or N), a carbonyl group, or —O—CO—C₁₋₅ alkylene-, wherein the alkylene can be optionally substituted by one or more substituents selected from a group consisting of alkyl, aromatic, anti-aromatic, cycloalkyl, acetyl, amino, hydroxyl, and thiol groups, or an alkane or alkene group, of which the main carbon chain has carbon atom selected from one to ten, such alkane or alkene group can be optionally substituted by one or more substituents selected from a group consisting of alkyl, aromatic, anti-aromatic, cyclic group, carbonyl, acetyl, keto, amino, hydroxyl, and hetero-atom (such as O, S, or N) etc.; or —X—NH— group can be denoted as an amino acid, whose main chain has 2 to 6 carbon atoms, such as glycine, alanine, valine, leucine, isoleucine; x and y each individually represent an integer value, x≥1, y≥x and x+y≥3 such as x+y≥10.

In some embodiments, the particles are detectable by a particle size analyzer, a transmission electron microscope, or a scanning electron microscope.

In some embodiments, the composition is an aqueous composition or an oil-in-water composition.

In some embodiments, the macromolecule is an amphiphilic compound and the hydrophilic-lipophilic balance value (HLB value) of the macromolecule is greater than 7.

In some embodiments, the macromolecule is an amphiphilic compound and the hydrophilic-lipophilic balance value (HLB value) of the macromolecule is greater than 10.

In some embodiments, the macromolecule is an amphiphilic compound and the hydrophilic-lipophilic balance value (HLB value) of the macromolecule ranges from 7 to 25.

In some embodiments, the macromolecule is an amphiphilic compound and the hydrophilic-lipophilic balance value (HLB value) of the macromolecule is 8, 9, 10, 11, 12, 13, 14, 15, 16, 17, 18, 19, 20, 21, 22, 23, or 24.

In some embodiments, the hydrophobic compound is an organic compound and the number of carbon ranges from 8 to 50.

In some embodiments, the hydrophobic compound is an organic compound and the number of carbon ranges from 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39.

In some embodiments, the hydrophobic compound is selected from the group consisting of steroids, steroid derivatives, saturated or unsaturated long chain fatty amines, and saturated or unsaturated long chain fatty amine derivatives, or any combination thereof.

In some embodiments, the hydrophobic compound is selected from the group consisting of cholesterol derivatives, octadecylamine, and octadecylamine derivatives, or any combination thereof.

In some embodiments, the macromolecule is a compound of formula (II):

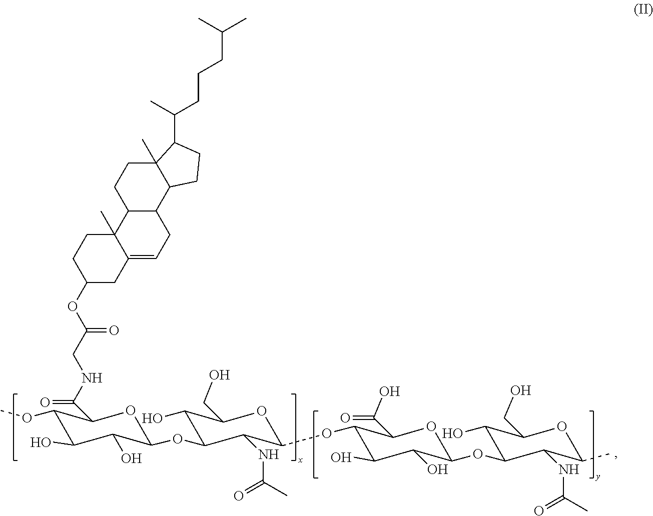

and wherein x and y each individually represent an integer value, x≥1, y≥x, and x+y≥3, such as x+y≥10.

In some embodiments, the macromolecule is a hyaluronic acid-cholesterol compound conjugate.

In some embodiments, the macromolecule is a compound of formula (III):

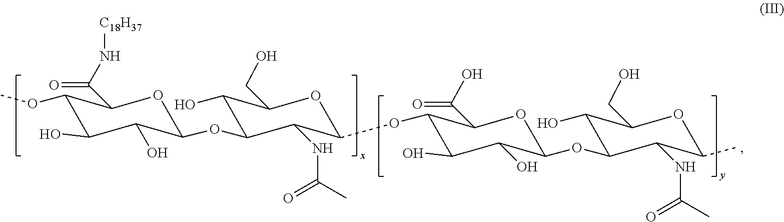

And wherein x and y each individually represent an integer value, x≥1, y≥x, and x+y≥3, such as x+y≥10.

In some embodiments, the macromolecule is a hyaluronic acid-octadecylamine conjugate.

In some embodiments, the terpenoid is liquid or solid at room temperature.

In some embodiments, the terpenoid is a cyclic compound or anon-cyclic compound.

In some embodiments, the carbon skeletons of the terpenoid includes (or consists of) 1˜20 unit(s) of isoprene carbon skeleton.

In some embodiments, the carbon skeletons of the terpenoid includes (or consists of) at least 21 units of isoprene carbon skeleton.

In some embodiments, the carbon skeletons of the terpenoid includes (or consists of) 2, 3, 4, 5, 6, 7, 8, 9, 10, 11, 12, 13, 14, 15, 16, 17, 18, or 19 units of isoprene carbon skeleton.

In some embodiments, the carbon skeletons of the terpenoid includes (or consists of) 1˜10 unit(s) of terpene(s) carbon skeleton.

In some embodiments, the carbon skeletons of the terpenoid includes (or consists of) at least 11 unit(s) of terpenes carbon skeleton.

In some embodiments, the carbon skeletons of the terpenoid includes (or consists of) 2, 3, 4, 5, 6, 7, 8, or 9 units of terpenes carbon skeleton.

In some embodiments, the terpenoid is an unsaturated terpenoid or a saturated terpenoid.

In some embodiments, the terpenoid is squalene, squalane, ocimene, or farnesene.

In some embodiments, the macromolecule has an average molecular weight of at least 10 kDa and less than or equal to 1000 kDa (10k˜1000k Da). In some embodiments, the macromolecule has an average molecular weight of 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, 95 kDa, 100 kDa, 105 kDa, 110 kDa, 115 kDa, 120 kDa, 125 kDa, 130 kDa, 135 kDa, 140 kDa, 145 kDa, 150 kDa, 155 kDa, 160 kDa, 165 kDa, 170 kDa, 175 kDa, 180 kDa, 185 kDa, 190 kDa, 195 kDa, 200 kDa, 205 kDa, 210 kDa, 215 kDa, 220 kDa, 225 kDa, 230 kDa, 235 kDa, 240 kDa, 245 kDa, 250 kDa, 255 kDa, 260 kDa, 265 kDa, 270 kDa, 275 kDa, 280 kDa, 285 kDa, 290 kDa, 295 kDa, 300 kDa, 305 kDa, 310 kDa, 315 kDa, 320 kDa, 325 kDa, 330 kDa, 335 kDa, 340 kDa, 345 kDa, 350 kDa, 355 kDa, 360 kDa, 365 kDa, 370 kDa, 375 kDa, 380 kDa, 385 kDa, 390 kDa, 395 kDa, 400 kDa, 405 kDa, 410 kDa, 415 kDa, 420 kDa, 425 kDa, 430 kDa, 435 kDa, 440 kDa, 445 kDa, 450 kDa, 455 kDa, 460 kDa, 465 kDa, 470 kDa, 475 kDa, 480 kDa, 485 kDa, 490 kDa, 495 kDa, 500 kDa, 505 kDa, 510 kDa, 515 kDa, 520 kDa, 525 kDa, 530 kDa, 535 kDa, 540 kDa, 545 kDa, 550 kDa, 555 kDa, 560 kDa, 565 kDa, 570 kDa, 575 kDa, 580 kDa, 585 kDa, 590 kDa, 595 kDa, 600 kDa, 605 kDa, 610 kDa, 615 kDa, 620 kDa, 625 kDa, 630 kDa, 635 kDa, 640 kDa, 645 kDa, 650 kDa, 655 kDa, 660 kDa, 665 kDa, 670 kDa, 675 kDa, 680 kDa, 685 kDa, 690 kDa, 695 kDa, 700 kDa, 705 kDa, 710 kDa, 715 kDa, 720 kDa, 725 kDa, 730 kDa, 735 kDa, 740 kDa, 745 kDa, 750 kDa, 755 kDa, 760 kDa, 765 kDa, 770 kDa, 775 kDa, 780 kDa, 785 kDa, 790 kDa, 795 kDa, 800 kDa, 805 kDa, 810 kDa, 815 kDa, 820 kDa, 825 kDa, 830 kDa, 835 kDa, 840 kDa, 845 kDa, 850 kDa, 855 kDa, 860 kDa, 865 kDa, 870 kDa, 875 kDa, 880 kDa, 885 kDa, 890 kDa, 895 kDa, 900 kDa, 905 kDa, 910 kDa, 915 kDa, 920 kDa, 925 kDa, 930 kDa, 935 kDa, 940 kDa, 945 kDa, 950 kDa, 955 kDa, 960 kDa, 965 kDa, 970 kDa, 975 kDa, 980 kDa, 985 kDa, 990 kDa, or 995 kDa. In some embodiments, the average molecular weight is a number-average molecular weight, as determined by gel filtration chromatography (GFC). In some embodiments, the average molecular weight is a weight-molecular weight, as determined by GFC (Please see: Bernice Yeung and Dale Marecak “Molecular weight determination of hyaluronic acid by gel filtration chromatography coupled to matrix-assisted laser desorption ionization mass spectrometry.” Journal of Chromatography A 85.2 (1999): 573-581).

In some embodiments, in the composition, at least 70% by molar ratio of the units per macromolecule on average are the first units.

In some embodiments, in the composition, the weight ratio of the terpenoid and the macromolecule ranges from 1:100 to 80:1.

In some embodiments, in the composition, the weight ratio of the terpenoid and the macromolecule is 1:100, 1:90, 1:80, 1:70, 1:60, 1:50, 1:40, 1:30, 1:20, 1:10, 1:1, 2:1, 3:1, 4:1, 5:1, 6:1, 7:1, 8:1, 9:1, 10:1, 12:1, 14:1, 16:1, 18:1, 20:1, 25:1, 30:1, 35:1, 40:1, 45:1, 50:1, 55:1, 60:1, 65:1, 70:1, 75:1, or 80:1.

In some embodiments, in the composition, the weight percentages of the terpenoid and macromolecule are respectively 0.01% to 40% and 0.001% to 3.0%, based on the total weight of the composition.

In some embodiments, in the composition, the weight percentage of the terpenoid is 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, or 39% based on the total weight of the composition.

In some embodiments, wherein in the composition, the weight percentage of the macromolecule is 0.01%, 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.51%, 0.52%, 0.53%, 0.54%, 0.55%, 0.56%, 0.57%, 0.58%, 0.59%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99%, 1.00%, 1.01%, 1.02%, 1.03%, 1.04%, 1.05%, 1.06%, 1.07%, 1.08%, 1.09%, 1.10%, 1.11%, 1.12%, 1.13%, 1.14%, 1.15%, 1.16%, 1.17%, 1.18%, 1.19%, 1.20%, 1.21%, 1.22%, 1.23%, 1.24%, 1.25%, 1.26%, 1.27%, 1.28%, 1.29%, 1.30%, 1.31%, 1.32%, 1.33%, 1.34%, 1.35%, 1.36%, 1.37%, 1.38%, 1.39%, 1.40%, 1.41%, 1.42%, 1.43%, 1.44%, 1.45%, 1.46%, 1.47%, 1.48%, 1.49%, 1.50%, 1.51%, 1.52%, 1.53%, 1.54%, 1.55%, 1.56%, 1.57%, 1.58%, 1.59%, 1.60%, 1.61%, 1.62%, 1.63%, 1.64%, 1.65%, 1.66%, 1.67%, 1.68%, 1.69%, 1.70%, 1.71%, 1.72%, 1.73%, 1.74%, 1.75%, 1.76%, 1.77%, 1.78%, 1.79%, 1.80%, 1.81%, 1.82%, 1.83%, 1.84%, 1.85%, 1.86%, 1.87%, 1.88%, 1.89%, 1.90%, 1.91%, 1.92%, 1.93%, 1.94%, 1.95%, 1.96%, 1.97%, 1.98%, 1.99%, 2.00%, 2.01%, 2.02%, 2.03%, 2.04%, 2.05%, 2.06%, 2.07%, 2.08%, 2.09%, 2.10%, 2.11%, 2.12%, 2.13%, 2.14%, 2.15%, 2.16%, 2.17%, 2.18%, 2.19%, 2.20%, 2.21%, 2.22%, 2.23%, 2.24%, 2.25%, 2.26%, 2.27%, 2.28%, 2.29%, 2.30%, 2.31%, 2.32%, 2.33%, 2.34%, 2.35%, 2.36%, 2.37%, 2.38%, 2.39%, 2.40%, 2.41%, 2.42%, 2.43%, 2.44%, 2.45%, 2.46%, 2.47%, 2.48%, 2.49%, 2.50%, 2.51%, 2.52%, 2.53%, 2.54%, 2.55%, 2.56%, 2.57%, 2.58%, 2.59%, 2.60%, 2.61%, 2.62%, 2.63%, 2.64%, 2.65%, 2.66%, 2.67%, 2.68%, 2.69%, 2.70%, 2.71%, 2.72%, 2.73%, 2.74%, 2.75%, 2.76%, 2.77%, 2.78%, 2.79%, 2.80%, 2.81%, 2.82%, 2.83%, 2.84%, 2.85%, 2.86%, 2.87%, 2.88%, 2.89%, 2.90%, 2.91%, 2.92%, 2.93%, 2.94%, 2.95%, 2.96%, 2.97%, 2.98%, or 2.99%, based on the total weight of the composition.

In some embodiments, wherein at least 50% (w/w) of terpenoid in the composition is encapsulated in the particles, based on the total content of terpenoid in the composition being 100% by mass. In some embodiments, wherein at least 55%, 60%, 65%, 70%, 75%, 80%, 85%, 90%, 95%, 98%, 99%, 99.5%, or 99.9% (w/w) of terpenoid in the composition is encapsulated in the particles, based on the total content of terpenoid in the composition being 100% by mass.

In some embodiments, wherein the composition comprising at least 1% (v/v) of terpenoid, based on the total volume of the composition. In some embodiments, wherein the composition comprising 1%˜40% (v/v) of terpenoid, based on the total volume of the composition. In some embodiments, wherein the composition comprising 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, or 39% (v/v) of terpenoid, based on the total volume of the composition.

In some embodiments, the compositions further comprise a solvent. In some embodiments, the solvent is water, normal saline, pure water, electrolyte water, glucose water, or other aqueous solution that could be used in the pharmaceutical injection. In some embodiments, in the composition, the weight percentage of the solvent is 57%, 58%, 59%, 60%, 61%, 62%, 63%, 64%, 65%, 66%, 67%, 68%, 69%, 70%, 71%, 72%, 73%, 7%, 75%, 76%, 77%, 78%, 79%, 80%, 81%, 82%, 83%, 84%, 85%, 86%, 87%, 88%, 89%, 90%, 91%, 92%, 93%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, based on the total weight of the composition. In some embodiments, in the composition, the volume percentage of the solvent is 57%, 58%, 59%, 60%, 61%, 62%, 63%, 64%, 65%, 66%, 67%, 68%, 69%0, 70% 71%, 72%, 73%, 7%, 75%, 76%, 77%, 78%, 79%, 80%, 81%, 82%, 83%, 84%, 85%, 86%, 87%, 88%, 89%, 90%, 91%, 92%, 93%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, based on the total volume of the composition.

In some embodiments, the solvent is water.

In some embodiments, the macromolecule is prepared by a method comprising mixing 0.0001˜2 equivalent of the hydrophobic compound with 1 equivalent hyaluronic acid.

In some embodiments, the macromolecule is prepared by a method comprising mixing 0.0001, 0.001, 0.01, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 equivalent of the hydrophobic compound with 1 equivalent hyaluronic acid.

In some embodiments, in the composition, the molar ratio of the first unit and the second unit per macromolecule on average ranges from 99:1 to 1:1.

In some embodiments, in the composition, the molar ratio of the first unit and the second unit per macromolecule on average is 98:1, 97:1, 96:1, 95:1, 94:1, 93:1, 92:1, 91:1, 90:1, 89:1, 88:1, 87:1, 86:1, 85:1, 84:1, 83:1, 82:1, 81:1, 80:1, 79:1, 78:1, 77:1, 76:1, 75:1, 74:1, 73:1, 72:1, 71:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, or 2:1.

In some embodiments, the diameter of the particles is 3˜1000 nm.

In some embodiments, the diameter of the particles is 5, 10, 15, 20, 25, 30, 35, 40, 45, 50, 55, 60, 65, 70, 75, 80, 85, 90, 95, 100, 120, 140, 150, 160, 180, 200, 220, 240, 150, 260, 280, 300, 320, 340, 350, 360, 380, 400, 420, 440, 450, 460, 480, 500, 520, 540, 550, 560, 580, 600, 620, 640, 650, 660, 680, 700, 720, 740, 750, 760, 780, 800, 820, 840, 850, 860, 880, 900, 920, 940, 950, 960, or 980 nm.

In some embodiments, the particles have a polydispersity index value (PDI) less than 0.4 to about 0.01.

In some embodiments, the particles have a polydispersity index value (PDI) less than 0.375, 0.35, 0.325, 0.3, 0.275, 0.25, 0.225, 0.2, 0.175, 0.15, 0.125, 0.1, 0.075, 0.05, or 0.025.

In some embodiments, provided herein are pharmaceutical compositions comprising the compositions provided herein.

In some embodiments, the pharmaceutical compositions further comprise a pharmaceutically acceptable carrier or excipient.

Provided herein are also uses of the compositions or the pharmaceutical compositions provided herein in stimulating an immune response in a subject.

Provided herein are also uses of the compositions or the pharmaceutical compositions provided herein in the treatment or prevention of a disease or disorder.

Provided herein are also methods of stimulating an immune response in a subject in need thereof, comprising administering the compositions or the pharmaceutical compositions provided herein to the subject.

Provided herein are also methods of treating or preventing a disease or disorder in a subject in need thereof, comprising administering the compositions or the pharmaceutical compositions provided herein to the subject.

In some embodiments, the pharmaceutical composition is a vaccine.

In some embodiments, the disease or disorder is a tumor or cancer.

In some embodiments, the tumor or cancer is a breast cancer.

In some embodiments, the disease or disorder is a pathogenic disease, HV or other viral infection, fungal infection, protozoan infection, or bacterial infection.

In some embodiments, the subject is a mammal, such as human.

Provided herein are also methods for increasing an immune response evoked by an antigenic component comprising administering the compositions or the pharmaceutical compositions provided herein with the antigenic component.

Provided herein are also kits (or vaccines) comprising the compositions or the pharmaceutical compositions provided herein with an antigenic component.

In some embodiments, the antigenic component is a target pathogen, target tumor cell, sub-portion of the target pathogen, sub-portion of the target tumor cell, pathogen antigen, cancer antigen, or any combination thereof.

In some embodiments, the antigenic component is selected from HER2, HER3, EGFR, VEGF, VEGFR2, CA-125, MUC series, p53, MAGE, NY-ESO-1, GAGE, BAGE, KRAS, NRAS, BCR-ABL translocation, ETV6, NPM/ALK, ALK, EBV LMP-1/LMP-2A, HPV E6/E7, HTLV-1 Tax, Melan A/MART-1, gp100, Tyrosinase, PSA, CEA, hTERT, p53, Survivin, WT1, cyclin B. Globo H, and SSEA series, or any combinations thereof.

### BRIEF DESCRIPTION OF THE DRAWINGS

FIG. 1A provides the results of structural identification performed for (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((tert-butoxycarbonyl)amino)acetate analyzed by 1H NMR (CDCl3, 400 MHz) and mass spectrums (ESI-MS).

FIG. 1B provides the results of structural identification performed for (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-aminoacetate analyzed by 1H NMR (CDCl3, 400 MHz) and mass spectrums (ESI-MS).

FIG. 1C provides the result of structural identification performed for HACH20 analyzed by 1H NMR spectrum (D2O/d6-DMSO, 400 MHz)

FIG. 2 provides the Visual appearance and TEM images of HA-derivatives and SQ@HA-derivatives in the present invention.

FIG. 3A-3B provide results which show the effect of oil@HACH on antigen presenting cells (APC) recruitment. FIG. 3A: dendritic cells recruitment; FIG. 3B: macrophages recruitment.

FIG. 4A-4B provide results which show the effect of reactants and product in the Squalene@HACH synthesis process on antigen presenting cells (APC) recruitment. FIG. 4A: dendritic cells recruitment; FIG. 4B: macrophages recruitment. Please note that the symbol “*” represents the animals in this group died during the experiment.

FIG. 5A-5B provide results which show the effect of the mixture comprising Squalene@HACH and targeted antigen on antigen presenting cells (APC) recruitment. FIG. 5A: dendritic cells recruitment; FIG. 5B: macrophages recruitment.

FIG. 6 provides result which shows the effect of the mixture comprising Squalene@HACH and targeted antigen on T cell-related cytokine production.

FIG. 7A-7B provide results from mouse model studies demonstrating the anti-tumor activities of WT-1+Squalene@HACH. FIG. 7A provides the tumor free rate curves. FIG. 7B provides the survival rate curve.

FIG. 8A-8B provide results which show the effect of reactants and product in the Squalene@HAODA synthesis process on antigen presenting cells (APC) recruitment. FIG. 8A: dendritic cells recruitment; FIG. 8B: macrophages recruitment.

### DETAILED DESCRIPTION OF THE INVENTION

PEGylated formulations have been wildly applied in food, cosmetics, medicine, and many other related fields causing the ubiquitous presence of PEG antibodies in the modem human body; however, these pre-existing anti-PEG antibodies can bind to PEGylated formulations to activate the complement system and release anaphylatoxins (C3a or C5a), which may further stimulate mast cells, basophils, and tissue macrophages to release secondary mediators that might elicit CARPA. (Please see: Mohamed, Marwa, et al. “PEGylated liposomes: immunological responses.” Science and Technology of Advanced Materials 20.1 (2019): 710-724)

Provided herein is a composition, such as SQ@HACH. Compared with a licensed squalene-based adjuvant MF59 containing PEG derivatives, the APC recruitment capabilities between the present invention and MF59 are equivalent; however, the present invention does not stimulate the production of anti-PEG antibodies, which means it does not trigger further immunogenic responses. This makes the present invention have the potential to be a lower allergic substitute material. Since vaccines are injected in vast amounts of healthy people rather than patients, more significant concern should be given to adjuvants to the health hazards.

Provided herein is an adjuvant composition comprising terpenoid and macromolecule; the terpenoid and macromolecule together form a plurality of particles; the macromolecule comprises: (1) at least one first unit, wherein the first unit is a disaccharide in which D-glucuronic acid is linked to N-acetylglucosamine; and (2) at least one second unit, wherein the second unit is a disaccharide-derivative in which D-glucuronic acid is linked to N-acetylglucosamine and a hydrophobic compound is attached to carbon 6 of said D-glucuronic acid; wherein the at least one first unit and the at least one second unit are linked to each other to form the macromolecule.

Before the present disclosure is further described, it is to be understood that the disclosure is not limited to the particular embodiments set forth herein, and it is also to be understood that the terminology used herein is for the purpose of describing particular embodiments, and is not intended to be limiting.

Unless otherwise defined herein, scientific and technical terms used in the present disclosures shall have the meanings that are commonly understood by those of ordinary skill in the art. Further, unless otherwise required by context, singular terms shall include pluralities and plural terms shall include the singular. Generally, nomenclatures used in connection with, and techniques of, organic chemistry, cell and tissue culture, molecular biology, immunology, microbiology, and chemistry and hybridization described herein are those well-known and commonly used in the art.

### 1.1 Composition

Provided herein are compositions comprising oil and macromolecule; the oil and macromolecule together form a plurality of particles; the macromolecule comprises: (1) at least one first unit, wherein the first unit is a first disaccharide; and (2) at least one second unit, wherein the second unit is a conjugation of a second disaccharide and a hydrophobic compound; wherein the at least one first unit and the at least one second unit are linked to each other to form the macromolecule. In some embodiments, the oil may be any vegetable oil, animal oil, mineral oil or synthetically prepared oil which can be metabolized by the body of the subject to which the adjuvant will be administered to the subject. In some embodiments, the oil component of this invention may be any long chain alkane, alkene or alkyne, or an acid or alcohol derivative thereof either as the free acid, its salt or an ester such as a mono-, or di- or triester, such as the triglycerides and esters of 1,2-propanediol or similar poly-hydroxy alcohols.

Provided herein are compositions comprising terpenoid and macromolecule; the terpenoid and macromolecule together form a plurality of particles; the macromolecule comprises: (1) at least one first unit, wherein the first unit is a disaccharide in which D-glucuronic acid is linked to N-acetylglucosamine; and (2) at least one second unit, wherein the second unit is a disaccharide-derivative in which D-glucuronic acid is linked to N-acetylglucosamine and a hydrophobic compound is attached to carbon 6 of said D-glucuronic acid; wherein the at least one first unit and the at least one second unit are linked to each other to form the macromolecule.

The term “terpenoid” as used herein refers to “an organic compound includes monoterpenes, sesquiterpene, diterpenes, or triterpene and is composed of linked isoprene units” or “a hydrocarbon that consist of terpenes attached to an oxygen-containing group” or “an organic compound includes monoterpenes, sesquiterpenes, diterpenes, triterpenes, tetraterpenes, or polyterpenes” or “an organic compound in which the carbon skeletons thereof is composed of at least one units of isoprene carbon skeleton” or “an organic compound that consist of terpenes attached to an oxygen-containing group”.

In some embodiments, the terpenoid is liquid or solid at room temperature. Preferably, in some embodiments, the terpenoid is liquid at room temperature. In some embodiments, the terpenoid is a cyclic compound or a non-cyclic compound.

In some embodiments, the terpenoid includes (or consists of) 1˜10 terpene(s); or the terpenoid includes (or consists of) at least 11 terpenes. Preferably, in some embodiments, the terpenoid includes (or consists of) 1˜10 terpene(s).

In some embodiments, the terpenoid is squalene, squalane, ocimene, or farnesene, other terpenoid, or any combination thereof.

The term “macromolecule” as used herein refers to “a molecule containing at least 100 atoms.”

In some embodiments, the macromolecule is a polymer. In some embodiments, the macromolecule is a hyaluronic acid derivative.

In some embodiments, the macromolecule is a hyaluronic acid-hydrophobic compound conjugate.

In some embodiments, the macromolecule is a compound of formula (I). In some embodiments, the macromolecule is a compound of formula (II) or (III).

In some embodiments, the terpenoid is an oil, and the macromolecule is an emulsifying agent; the composition is an oil-in-water composition.

In some embodiments, the hydrophilic-lipophilic balance value (HLB value) of the macromolecule is greater than 7. In some embodiments, the hydrophilic-lipophilic balance value (HLB value) of the macromolecule is greater than 10. In some embodiments, the hydrophilic-lipophilic balance value (HLB value) of the macromolecule is ranges from 7 to 20.

In some embodiments, the hydrophilic-lipophilic balance value (HLB value) of the macromolecule is 8, 9, 10, 11, 12, 13, 14, 15, 16, 17, 18, or 19.

In some embodiments, the particle comprises (a) a microstructure formed by the macromolecule and (b) the terpenoid encapsulated in the microstructure.

In some embodiments, the particles are detectable by a particle size analyzer, a transmission electron microscope, or a scanning electron microscope.

In some embodiments, diameter of the particles is 3˜1000 nm.

In some embodiments, diameter of the particles is 5, 10, 15, 20, 25, 30, 35, 40, 45, 50, 55, 60, 65, 70, 75, 80, 85, 90, 95, 100, 120, 140, 150, 160, 180, 200, 220, 240, 150, 260, 280, 300, 320, 340, 350, 360, 380, 400, 420, 440, 450, 460, 480, 500, 520, 540, 550, 560, 580, 600, 620, 640, 650, 660, 680, 700, 720, 740, 750, 760, 780, 800, 820, 840, 850, 860, 880, 900, 920, 940, 950, 960, or 980 nm.

In some embodiments, the particles have a polydispersity index value (PDI) less than 0.4 to about 0.01.

In some embodiments, the particles have a polydispersity index value (PDI) less than 0.375, 0.35, 0.325, 0.3, 0.275, 0.25, 0.225, 0.2, 0.175, 0.15, 0.125, 0.1, 0.075, 0.05, or 0.025.

The term “first unit” as used herein refers to “a disaccharide composed of a D-glucuronic acid and a N-acetylglucosamine, wherein the D-glucuronic acid is linked to the N-acetylglucosamine”.

In some embodiments, the D-glucuronic acid and N-acetylglucosamine in the first unit are linked by a β-(1,3) bond.

The term “second unit” as used herein refers to “a disaccharide-derivative composed of a D-glucuronic acid, a N-acetylglucosamine, and a hydrophobic compound; wherein the D-glucuronic acid is linked to the N-acetylglucosamine, and the hydrophobic compound is attached to carbon 6 of the D-glucuronic acid”.

In some embodiments, the D-glucuronic acid and N-acetylglucosamine in the second unit are linked by a β-(1,3) bond.

In some embodiments, the hydrophobic compound is selected from the group consisting of steroids, steroid derivatives, saturated or unsaturated long chain fatty amines, and saturated or unsaturated long chain fatty amine derivatives, or any combination thereof.

In some embodiments, the hydrophobic compound is selected from the group consisting of cholesterol derivatives, octadecylamine, and octadecylamine derivatives, or any combination thereof.

In some embodiments, the macromolecule composed of one first unit and at least two second units, and these units are linked to each other to form the macromolecule. In some embodiment, the macromolecule composed of at least two first units and one second unit, and these units are linked to each other to form the macromolecule. In some embodiment, macromolecule composed of at least two first units and at least two second units, and these units are linked to each other to form the macromolecule.

In some embodiments, two first units located in a portion of the macromolecule are linked to each other to form the portion of the macromolecule. In some embodiments, two second units located in a portion of the macromolecule are linked to each other to form the portion of the macromolecule. In some embodiments, a first unit and a second unit located in a portion of the macromolecule are linked to each other to form the portion of the macromolecule.

In some embodiments, the units (the first unit(s) and/or the second unit(s)) are linked to each other by a β-(1,4) bond to form the macromolecule.

In some embodiments, the macromolecule is prepared by a method comprising mixing 0.0001˜2.0 equivalent of the hydrophobic compound with 1 equivalent of hyaluronic acid. For example, in some embodiments, 0.1 equivalent of (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-aminoacetate (a hydrophobic compound) and 1 equivalent of hyaluronic acid are mixed for HACH10 preparation; 0.2 equivalent of cholesterol-glycine-NH₂ and 1 equivalent of hyaluronic acid are mixed for HACH20 preparation; 0.3 equivalent of (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-aminoacetate and 1 equivalent of hyaluronic acid are mixed for HACH30 preparation; 0.05 equivalent of (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-aminoacetate and 1 equivalent of hyaluronic acid are mixed for HACH5 preparation; 0.25 equivalent of (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-aminoacetate and 1 equivalent of hyaluronic acid are mixed for HACH25 preparation. In some embodiments, 0.05 equivalent of octadecylamine (a hydrophobic compound) and 1 equivalent of hyaluronic acid are mixed for HAODA5 preparation; 0.15 equivalent of octadecylamine and 1 equivalent of hyaluronic acid are mixed for HAODA15 preparation; 0.25 equivalent of octadecylamine and 1 equivalent of hyaluronic acid are mixed for HAODA25 preparation.

In the composition, the molar ratio of the first unit and the second unit per macromolecule on average ranges from 99:1 to 1:1. In some embodiments, in the composition, the molar ratio of the first unit and the second unit per macromolecule on average is 98:1, 97:1, 96:1, 95:1, 94:1, 93:1, 92:1, 91:1, 90:1, 89:1, 88:1, 87:1, 86:1, 85:1, 84:1, 83:1, 82:1, 81:1, 80:1, 79:1, 78:1, 77:1, 76:1, 75:1, 74:1, 73:1, 72:1, 71:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 5:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, or 2:1.

### 1.2 Pharmaceutical Composition

In some embodiments, provided herein are pharmaceutical compositions comprising the compositions provided herein.

In some embodiments, the pharmaceutical composition is a vaccine. In some embodiments, the pharmaceutical composition is a natural vaccine, a synthetic vaccine, or a recombinant vaccine. In some embodiments, the pharmaceutical composition is a DNA vaccine or a RNA vaccine. In some embodiments, the pharmaceutical composition is an inactivated vaccine, a live-attenuated vaccine, a messenger RNA (mRNA) vaccine, a subunit, recombinant, polysaccharide, and conjugate vaccine, a toxoid vaccine, or a viral vector vaccine.

In some embodiments, the compositions or the pharmaceutical compositions provided herein further comprises an active ingredient or a targeted antigen (or antigen, or antigenic component). In some embodiments, the active ingredient or the targeted antigen (or antigen, or antigenic component) is for treating a disease or a disorder. In some embodiments, the active ingredient or the targeted antigen (or antigen, or antigenic component) is for treating cancer, and the active ingredient or the targeted antigen (or antigen, or antigenic component) is WT1 protein, OVA, or a combination thereof.

As a person of ordinary skill in the art would understand, the term “pharmaceutically acceptable carrier” or “pharmaceutically acceptable excipient” refers to, for example, a material that is suitable for drug administration to an individual along with an active agent or targeted molecule or antigen without causing undesirable biological effects or interacting in a deleterious manner with any of the other components of the pharmaceutical composition.

In some embodiments, the pharmaceutical composition is an aqueous formulation. Such a formulation is typically a solution or a suspension, but can also include colloids, dispersions, emulsions, and multi-phase materials. The term “aqueous formulation” is defined as a formulation comprising at least 50% w/w water.

### 1.3 Methods of Uses

In some embodiments, provided herein are uses of the composition or the pharmaceutical composition provided herein in the treatment or prevention of a disease or disorder.

In some embodiments, provided herein are methods of stimulating an immune response in a subject, comprising administering the composition or the pharmaceutical composition provided herein to the subject.

In some embodiments, the immune response is an antibody response (humoral immunity response), antigen-specific T-cell response (cell-mediated immunity response), innate immune response, or any combination thereof. In some embodiments, the antigen-specific T-cell response is CD8⁺ T-cell response and/or CD4⁺ T-cell mediated immune response. In some embodiments, the immune response is T helper cell immune response. In some embodiments, the T helper cell immune response is a T helper 1 response or a T helper 2 response or T helper 17 response or both. In some embodiments, the immune response is recruitment of dendritic cells (CD11c⁺) or recruitment of macrophages (CD11b⁺). In some embodiments, the immune response is T cell immunity, CD4⁺ T-cell response, antigen-specific T cell immunity, or Th1/Th2/Th17-related cytokines production.

The term “treat” as used herein in connection with a disease or a condition, or a subject having a disease or a condition refer to an action that suppresses, eliminates, reduces, and/or ameliorates a symptom, the severity of the symptom, and/or the frequency of the symptom associated with the disease or disorder being treated. For example, when used in reference to a cancer or tumor, the term “treat” refer to an action that reduces the severity of the cancer or tumor, or retards or slows the progression of the cancer or tumor, including (a) inhibiting the growth, or arresting development of the cancer or tumor, (b) causing regression of the cancer or tumor, or (c) delaying, ameliorating or minimizing one or more symptoms associated with the presence of the cancer or tumor.

The term “administer” as used herein refer to the act of delivering, or causing to be delivered, a therapeutic or a pharmaceutical composition to the body of a subject by a method described herein or otherwise known in the art. The therapeutic can be a compound, a polypeptide, an antibody, a cell, or a population of cells. Administering a therapeutic or a pharmaceutical composition includes prescribing a therapeutic or a pharmaceutical composition to be delivered into the body of a subject.

The terms “effective amount” or “therapeutically effective amount” as used herein refer to the administration of an agent to a subject, either alone or as a part of a pharmaceutical composition and either in a single dose or as part of a series of doses, in an amount that is capable of having any detectable, positive effect on any symptom, aspect, or characteristics of a disease, disorder or condition when administered to the subject. The therapeutically effective amount can be ascertained by measuring relevant physiological effects. The exact amount required vary from subject to subject, depending on the age, weight, and general condition of the subject, the severity of the condition being treated, the judgment of the clinician, and the like. An appropriate “effective amount” in any individual case can be determined by one of ordinary skill in the art using routine experimentation.

The term “subject” as used herein refers to any animal (e.g., a vertebrate). The subjects include, but are not limited to, humans, non-human primates, simians, canines, felines, rodents, and the like, which is to be the recipient of a particular treatment. A subject can be a human. A subject can be a mammal. A subject can be a farm animal. A subject can be a pet. A subject can have a particular disease or condition.

The patient or subject to be treated can be a human patient with a disease or disorder described herein. In some embodiments, the subject is a cancer patient. In some embodiments, the subject is a virus-infected patient, bacteria-infected patient, fungi-infected patient, protozoan-infected patient. In some embodiments, the subject has and/or is being treated for a cancer or tumor.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### 1.4 Preparation Methods of Composition

### 1.4.1 Preparation Methods of Macromolecules

Equation (a) provides the synthetic pathway of some macromolecules (refer to amphiphilic HA-derivatives or amphiphilic HA-hydrophobic compound conjugates) of the present invention.

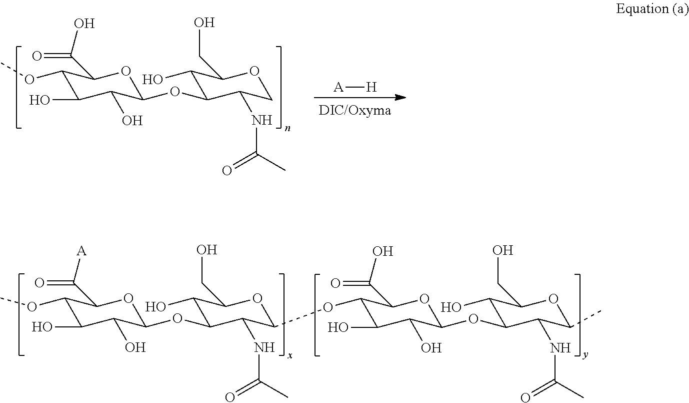

In Equation (a), the compound A-H is a hydrophobic compound, and A group is R—NH— or R—X—NH— that is conjugated to the carboxylic group of the hyaluronic acid to form the macromolecule (refer to amphiphilic HA-derivatives or amphiphilic HA-hydrophobic compound conjugates) comprising first unit(s) and second unit(s) in some embodiments of the present invention; wherein R group is a hydrophobic group derived from the hydrophobic compound, X denotes a hetero-atom (such as O, S, or N), a carbonyl group, or —O—CO—C₁₋₅ alkylene-, wherein the alkylene can be optionally substituted by one or more substituents selected from a group consisting of alkyl, aromatic, anti-aromatic, cycloalkyl, acetyl, amino, hydroxyl, and thiol groups, or an alkane or alkene group, of which the main carbon chain has carbon atom selected from one to ten, such alkane or alkene group can be optionally substituted by one or more substituents selected from a group consisting of alkyl, aromatic, anti-aromatic, cyclic group, carbonyl, acetyl, keto, amino, hydroxyl, and hetero-atom (such as O, S, or N) etc.; or —X—NH— group can be denoted as an amino acid, whose main chain has 2 to 6 carbon atoms, such as glycine, alanine, valine, leucine, isoleucine; x and y each individually represent an integer value, n=x+y, x≥1, y≥x, and x+y≥3, such as x+y≥10.

Inventor of the present invention further provides the synthetic method of these macromolecules (refer to amphiphilic HA-derivatives or amphiphilic HA-hydrophobic compound conjugates), including the following steps:

1.0 equivalent of hyaluronic acid is first dissolved in a co-solvent which is a mixture of water and an organic solvent (such as DMSO). A mixture containing 1.1 equivalent of ethyl cyanohydroxyiminoacetate and appropriate equivalent (e.g. according the reactivity of expectedly substituted compound) of R—X—NH₂ is dissolved in an organic solvent (such as DMSO) and then the resulting solution is added into the hyaluronic acid solution (HA solution). The mixed solution is slowly added by 2.0 equivalent of N,N′-diisopropylcarbodiimide and stirred for 24 hours. The obtained solution is transferred into a suitable molecular weight cut off (MWCO) dialysis bag (such as 3500 Da MWCO dialysis bag) and purified by sequential dialysis against co-solvent (50/50, v/v), 0.3 M NaCl aqueous solution, and pure water. Finally, water is removed from the dialyzed product solution by freeze-drying to obtain the macromolecule which has a molecule weight higher than MWCO (such as the macromolecule which has a molecule weight higher than 3500 Da; please note that the molecule weight of a hyaluronic acid consisting of 10 disaccharide units is about 3800 Da).

In Equation (a), hydrophobic compound R is (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-aminoacetate or (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-aminopropanoate or (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-aminopropanoate, or (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-aminobutanoate or (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl amino acid ester or octan-1-amine or nonan-1-amine or decan-1-amine or undecan-1-amine or dodecan-1-amine or tridecan-1-amine or tetradecan-1-amine or pentadecan-1-amine or hexadecan-1-amin or heptadecan-1-amine or octadecan-1-amine or saturated long chain fatty amines consisting of carbon number more than 19 or unsaturated octan-1-amine or unsaturated nonan-1-amine or unsaturated decan-1-amine or unsaturated undecan-1-amine or unsaturated dodecan-1-amine or unsaturated tridecan-1-amine or unsaturated tetradecan-1-amine or unsaturated pentadecan-1-amine or unsaturated hexadecan-1-amin or unsaturated heptadecan-1-amine or unsaturated octadecan-1-amine or unsaturated long chain fatty amines consisting of carbon number more than 19 or saturated long chain fatty amines or saturated long chain fatty amine derivatives or unsaturated long chain fatty amine derivatives, or any combination thereof.

### 1.4.2 Preparation Methods of Oil@Macromolecule Particles

In some embodiments, an oil and the Macromolecule prepared by the method as described in section 1.4.1 is added into an aqueous solution (such as sodium citrate solution) to obtain a resulting solution which is the composition comprising oil@macromolecule particles.

In some embodiments, an oil and the Macromolecule prepared by the method as described in section 1.4.1 is added into an aqueous solution (such as sodium citrate solution) to obtain a resulting solution, and the resulting solution is pre-mixed in a test tube rotator and then homogenized through a high-pressure microfluidizer to obtain a resulting solution which is the composition comprising oil@macromolecule particles.

In some embodiments, the oil is terpenoid, and the oil@macromolecule particles are terpenoid@macromolecule particles.

To evaluate the particle size and stability of the oil@macromolecule particles, the particle size and polydispersity index (PDI) of oil@macromolecule particles in the composition are measured by dynamic light scattering or particle size analyzer. The oil@macromolecule particles were stained by PTA negative staining and observed by transmission electron microscopy.

### EXPERIMENTS

The examples provided below are for purposes of illustration only, which are not intended to be limiting unless otherwise specified. Thus, the invention should in no way be construed as being limited to the following examples, but rather, should be construed to encompass any and all variations which become evident as a result of the teaching provided herein.

### Experiment 1: Composition Comprising Oil@HACH Particles

### Experiment 1-1 Preparation of Composition Comprising Oil@HACH Particles

In this embodiment, hyaluronic acid-cholesterol conjugate (HACH), which is a macromolecule of the present invention, was used.

Equations (b)˜(d) provide the synthetic pathways of hyaluronic acid-cholesterol conjugate (HACH) of the present invention.

Equation (b) Synthesis route of (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((tert-butoxycarbonyl)amino)acetate; (Modification of cholesterol):

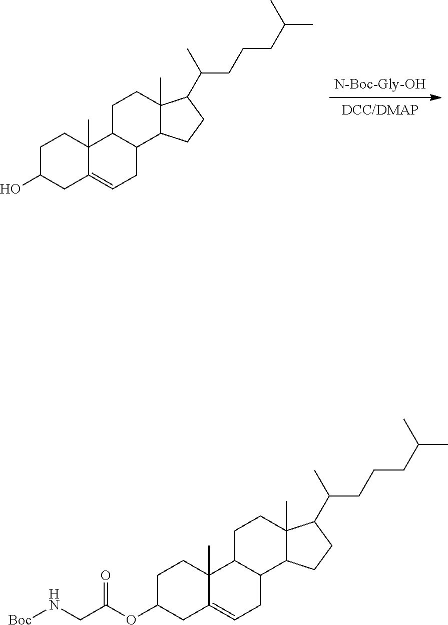 [SMILES: CC(C)CCCC(C)C1CCC2C3CC=C4CC(O)CCC4(C)C3CCC12C ; CC(C)CCCC(C)C1CCC2C3CC=C4CC(OC(=O)CNC(=O)OC(C)(C)C)CCC4(C)C3CCC12C]

Equation (c) Synthesis route of (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-aminoacetate; (Deprotection of (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((tert-butoxycarbonyl)amino)acetate):

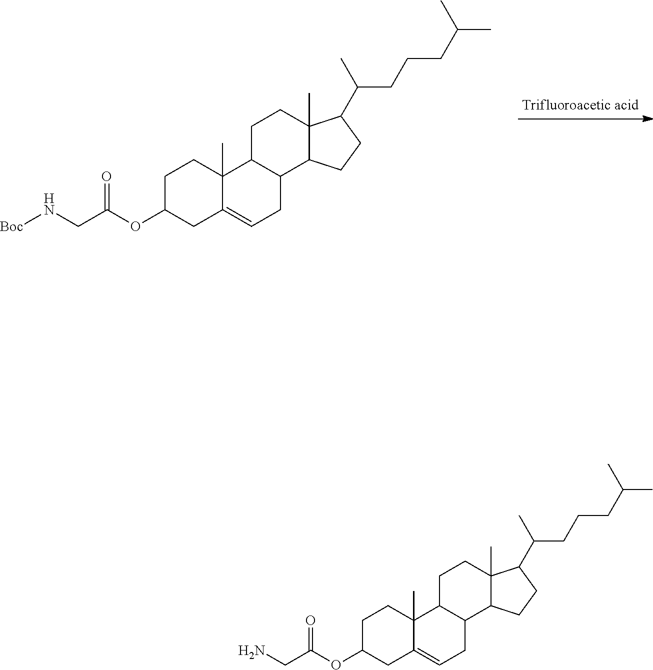 [SMILES: CC(C)CCCC(C)C1CCC2C3CC=C4CC(OC(=O)CNC(=O)OC(C)(C)C)CCC4(C)C3CCC12C ; CC(C)CCCC(C)C1CCC2C3CC=C4CC(OC(=O)CN)CCC4(C)C3CCC12C]

Equation (d) Synthesis route of HACH; (Conjugation of HA and (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-aminoacetate):

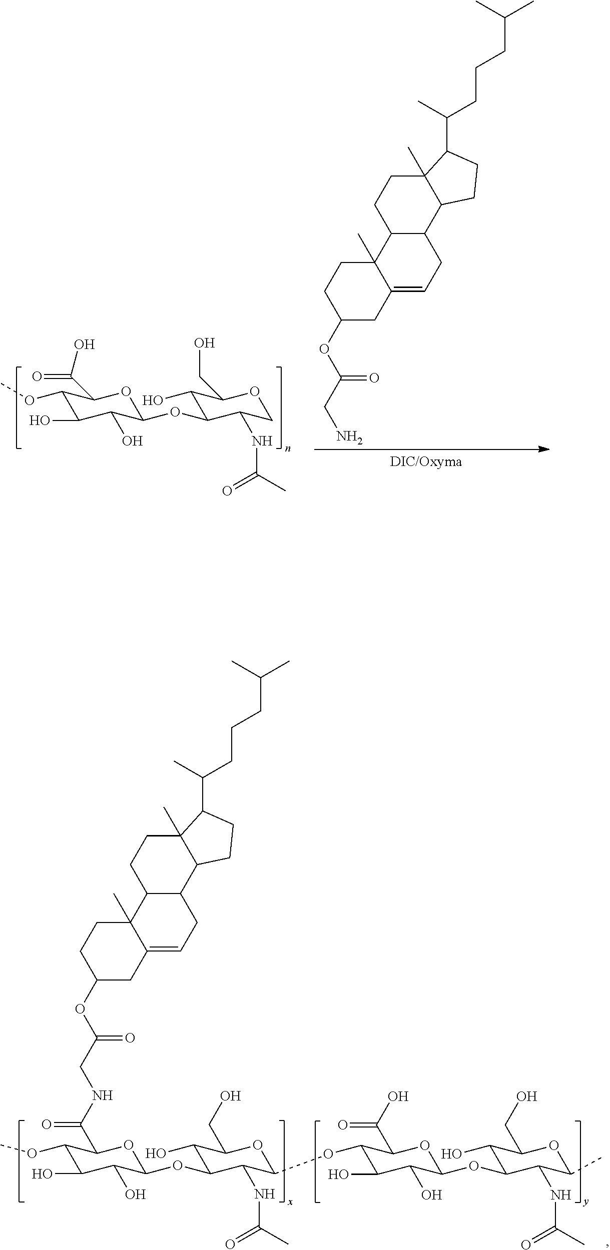

and wherein n, x, and y each individually represent an integer value, n=x+y, x≥1, y≥x, and x+y≥3, such as x+y≥10.

### Experiment 1-1-1 Synthesis route of (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((tert-butoxycarbonyl)amino)acetate; (Modification of cholesterol)

Please refer to equation (b), in the first step for the preparation of HACH, cholesterol was modified with Boc-Gly-OH by DCC/DMAP esterification to obtain (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((tert-butoxycarbonyl)amino)acetate. The detailed process in this first step comprises:

1000 mg (2.59 mmol) of cholesterol and 480 mg (2.74 mmol) of Boc-Gly-OH were dissolved in 125 mL of DCM, and then 1070 mg (5.18 mmol) of DCC and 372 mg (3.04 mmol) of DMAP were added into the mixture solution and reacted for 12 h under a N₂ atmosphere at room temperature. The reaction was traced by thin layer chromatography (TLC) to confirm that the coupling reaction had completed. After removing the solvent by rotary evaporation, the residues were purified by silica gel chromatography with a mobile phase mixture of hexane/acetone at 3/1. The structure of the product was identified by ¹H-NMR spectroscopy (Agilent Technologies 400 MHz NMR, Santa Clara, CA, USA) and mass spectroscopy (TSQ Altis™ Triple Quadrupole Mass Spectrometer, Thermo Fisher Scientific, Waltham, MA, USA) to confirm the product in this first step is (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((tert-butoxycarbonyl)amino)acetate. ¹H-NMR (400 MHz, CDCl₃) δ 5.37 (d, J=4.4 Hz, 1H), 4.99 (s, 1H), 4.68 (m, 1H), 3.87 (d, J=5.3 Hz, 2H), 2.33 (d, J=7.8 Hz, 2H), 2.01 (m, 2H), 1.95 (t, J=4.5 Hz, 1H), 1.90-1.77 (m, 3H), 1.63-1.41 (m, 9H), 1.45 (s, 9H), 1.40-1.23 (m, 5H), 1.20-1.03 (m, 8H), 1.01 (s, 3H), 0.91 (d, J=6.6 Hz, 3H), 0.86 (dd, J=6.6, 1.6 Hz, 6H), 0.67 (s, 3H); ESI-MS: C₃₄H₅₈NO₄⁺ [M+H]⁺ 543.9 m/z.

### Experiment 1-1-2 Synthesis route of (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-aminoacetate (Deprotection of (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((tert-butoxycarbonyl)amino)acetate)

Please refer to equation (c), in the second step for the preparation of HACH, the Boc group was removed under acidic conditions by Trifluoroacetic acid (TFA) to produce (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-aminoacetate. The detailed process in this second step comprises: [00172]500 mg (0.92 mmol) of (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((tert-butoxycarbonyl)amino)acetate was dissolved in 2 mL of DCM in an ice bath, and then 2 mL of Trifluoroacetic acid (TFA) was added into the solution and stirred for 3 h under a N₂ atmosphere at room temperature. The reaction was traced by TLC to check the completion of de-protection of Boc group. After de-protection, the mixture was neutralized with saturated NaHCO₃ aqueous solution and the precipitate was filtered out and dried in vacuum to obtain the (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-aminoacetate white powder. The structure of (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-aminoacetate was identified by ¹H-NMR spectroscopy (Agilent Technologies 400 MHz NMR) and mass spectroscopy (TSQ Altis™ Triple Quadrupole Mass Spectrometer). ¹H-NMR (400 MHz, CDCl₃) δ 5.37 (broad s, 1H), 4.68 (m, 1H), 3.79 (broad s, 2H), 2.33 (m, 2H), 2.01 (m, 2H), 1.95 (t, J=4.5 Hz, 1H), 1.90-1.77 (m, 3H), 1.63-1.41 (m, 7H), 1.40-1.23 (m, 6H), 1.20-1.03 (m, 7H), 1.01 (s, 3H), 0.91 (d, J=6.5 Hz, 3H), 0.86 (d, J=6.5 Hz, 6H), 0.67 (s, 3H); ESI-MS: C₂₉H₅₀NO₂⁺ [M+H]⁺ 444.2 m/z.

### Experiment 1-1-3 Synthesis route of HACH (Conjugation of HA and (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-aminoacetate)

Please refer to equation (d), in the final step for the preparation of HACH, (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-aminoacetate was conjugated to the carboxylic group of HA by DIC/Oxyma activation to form the HACH. In this step, HACH with a glycine linker was synthesized by using DIC/oxyma as an amide coupling agent to graft (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-aminoacetate onto HA in a DMSO/H₂O co-solvent system. After dialysis and lyophilization, the cotton-like HACH product was obtained. The detailed process in this final step comprises:

500 mg (1.25 mmol, 1.0 eq.) of hyaluronic acid (100 kDa) was first dissolved in a mixture of 70 mL of water and 90 mL of DMSO. A mixture containing 250 mg (1.11 mmol) of Oxyma and 113 mg (0.25 mmol, 0.2 eq.) of (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-aminoacetate was dissolved in 10 mL of DMSO and then the resulting solution was added into the hyaluronic acid solution (HA solution). The mixed solution was slowly added by 405 μL (2.58 mmol) of DIC and stirred for 24 h. The obtained solution was transferred into a 3500-MWCO dialysis bag (3500 Da molecular weight cut off dialysis bag) and purified by sequential dialysis against DMSO/water (50/50, v/v), 0.3 M NaCl aqueous solution, and pure water. Finally, water was removed from the dialyzed product solution by freeze-drying to obtain HACH20 (the obtained HACH20 would has a molecule weight higher than molecular weight cut off (MWCO), that is, a molecule weight higher than 3500 Da; please note that the molecule weight of a hyaluronic acid consist of 10 disaccharide units is about 3800 Da). The different DS % of HACH was synthesized by adding corresponding equivalents of (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-aminoacetate; HACH10 and HACH30 means 0.1 and 0.3 equivalent of (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-aminoacetate for the HA conjugation, respectively. The conjugation ratio (DS %) of HACH was shown in Table 1 and determined by elemental analysis (Elementarvario EL cube, Langenselbold, Hesse, Germany).

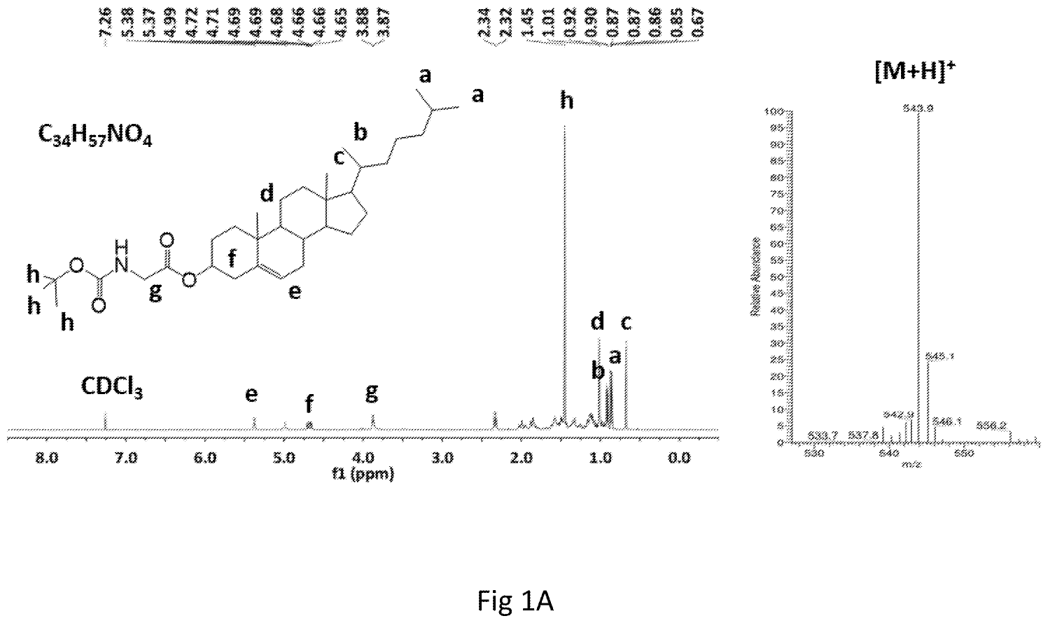

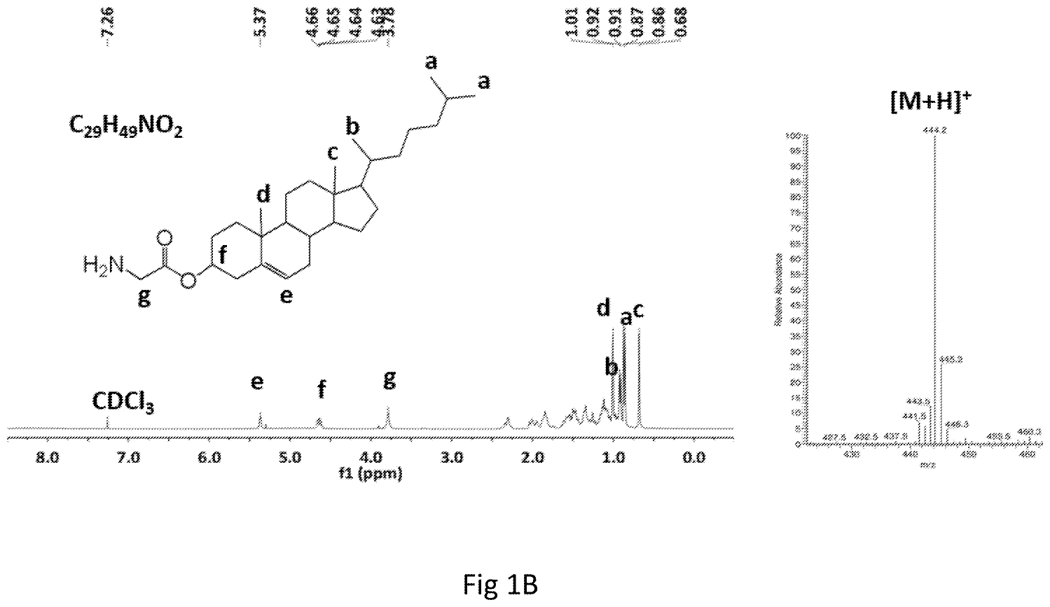

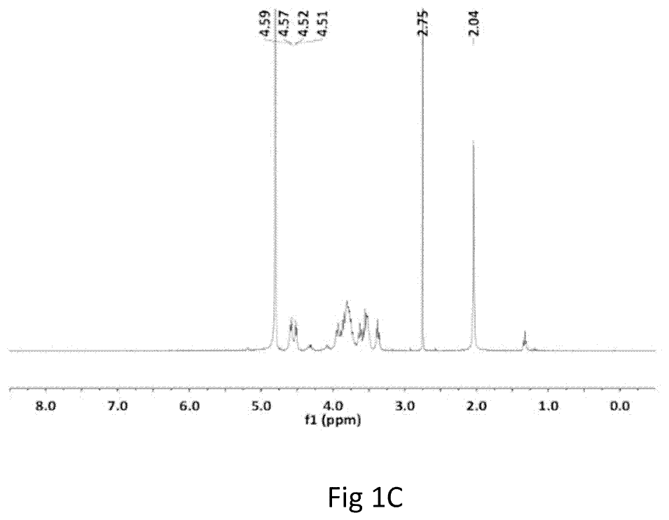

Please refer to FIGS. 1A to 1C. FIGS. 1A to 1B are the results of structural identification performed for (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((tert-butoxycarbonyl)amino)acetate (FIG. 1A) and (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-aminoacetate (FIG. 1B) analyzed by ¹H NMR (CDCl₃, 400 MHz) and mass spectrums (ESI-MS); FIG. 1C is the result of structural identification performed for HACH20 analyzed by ¹H NMR spectrum (D₂O/d6-DMSO, 400 MHz).

As shown in FIG. 1, the characteristic peaks of cholesterol (methyl groups at 0.67, 0.86, 0.91, and 1.01 ppm) and tert-butyl group (Peak h, 1.45 ppm, singlet, 9H) were observed in (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((tert-butoxycarbonyl)amino)acetate (FIG. 1A) and the tert-butyl group of Boc (tert-butyloxycarbonyl) peak at 1.45 ppm disappeared after Trifluoroacetic acid (TFA) incubation (FIG. 1B), indicating a successful de-protection process for obtaining (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-aminoacetate. The molecular weight of (8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-aminoacetate was also confirmed by mass spectroscopy.

The DS % of HACH was analyzed by an elemental analyzer (EA) and calculated using the following formula (IV):

$$DS = \frac{{R}_{y} - {R}_{0}}{{R}_{100} - {R}_{0}} \times 100 {\%}_{↵} ,$$

where R_{y} is the C/N ratio of HACH, and R₀ and R₁₀₀ represent the C/N ratios of unmodified HA and theoretical completely modified HACH (100%), respectively. Actual analyzed methods are known, or will be apparent, to those skilled in the art.

Please refer to Table 1. The theoretical carbon to nitrogen (C/N) ratio of HA alone or HACH with 100% degree of substitution (DS) was 14 and 21.5, respectively. To determine the DS % of cholesterol of the HACH examples in this experiment, inventors of the present invention employed EA to investigate the C/N ratio of free HA and HACH and then calculated the DS ratio of HACH using Formula (IV). As shown in Table 1, the actual C/N of HA measured by EA was 14.01, this value was consistent with the theoretical C/N ratio of HA and indicated the reliability of EA analysis in the determination of C/N ratio of HA or HA-derivatives. The C/N of HACH5 was 14.36 analyzing by EA and the DS ratio was calculated as 4.8% that was closed to the estimated DS ratio of 5%. HACH10 revealed a C/N ratio of 14.66, and DS ratio of 8.8% even though the DS ratio of HACH was estimated at 10% for the conjugation process. If the estimated DS ratios were increased to 20%, 25%, and 30%, the measured C/N ratios were respectively increased to 14.97, 15.27, and 15.56, and the DS ratios correspondingly were 12.9%, 16.9%, 20.8%.

TABLE 1
Calculation of DS % of HACH by elemental analyzer

| | C atom(mole %)^{a} | N atom(mole %)^{a} | C/N ^{b} | DS(%) ^{c} |
|---|---|---|---|---|
| HA | 3.125 | 0.223 | 14.01 | — |
| 100k-HACH5 | 3.159 | 0.220 | 14.36 | 4.8 |
| 100k-HACH10 | 3.314 | 0.226 | 14.66 | 8.8 |
| 100k-HACH20 | 3.143 | 0.210 | 14.97 | 12.9 |
| 100k-HACH25 | 3.222 | 0.211 | 15.27 | 16.9 |
| 100k-HACH30 | 3.361 | 0.216 | 15.56 | 20.8 |

Moreover, the hydrophilic-lipophilic balance value (HLB value) of amphiphilic HA-derivatives was calculated according to the Griffin's method and expressed as following formula (V):

$${HLB}_{HA - derivatives} = 20 \times ( 1 - DS )$$

Please refer to Table 2. As shown in Table 2, the HLB value of the HACH examples in this experiment ranges from 15 to 20.

TABLE 2
Calculation of HLB value of amphiphilic HA-derivatives

| | DS(%) | HLB value |
|---|---|---|
| HA | 0 | 20.0 |
| 100k-HACH5 | 4.8 | 19.0 |
| 100k-HACH10 | 8.8 | 18.2 |
| 100k-HACH20 | 12.9 | 17.4 |
| 100k-HACH25 | 16.9 | 16.6 |
| 100k-HACH30 | 20.8 | 15.8 |

### Experiment 1-1-4 Preparation of the Composition Comprising Oil@HACH Particles

In this experiment, different kinds of oils were used to prepare compositions comprising oil@HACH particles. These oils were:
(1) paraffin oil, which is a mineral oil.
(2) squalane, which is a saturated terpenoid consisting of 3 terpenes (a triterpene) and is classified as a saturated triterpene.
(3) squalene, which is an unsaturated terpenoid consisting of 3 terpenes (a triterpene) and is classified as an unsaturated triterpene.
(4) ocimene, which is a terpenoid consisting of 1 terpene (a monoterpene) and is classified as a monoterpene.
(5) famesene, which is a terpenoid consisting of 1.5 terpenes (a sesquiterpene) and is classified as a sesquiterpene.

100 mg of HACH20 (the final concentration in the composition is 1% (w/v)) was dissolved in 9.5 mL of sodium citrate solution (pH=6.5, 10 mM), and then 500 μL of oil (the final concentration in the composition is 5% (v/v)) was added into the HACH solution. The resulting solution was pre-mixed in high shear mixer for 10 mins and then homogenized through a high-pressure homogenizer to obtain compositions comprising different oil@HACH particles including:
(A) Composition comprising Paraffin oil@HACH particles (Paraffin oil@HACH emulsion);
(B) Composition comprising Squalane@HACH particles (Squalane@HACH emulsion);
(C) Composition comprising Squalene@HACH particles (Squalene@HACH emulsion);
(D) Composition comprising Ocimene@HACH particles (Ocimene@HACH emulsion); and
(E) Composition comprising Famesene@HACH particles (Famesene@HACH emulsion).

To evaluate the particle size and stability of oil@HACH particles, aliquots of the compositions comprising oil@HACH particles were loaded in 1.5-mL Eppendorf tubes and stored separately at 4° C. to mimic the stored condition of normal vaccine. At predetermined time points, the appearance and particle sizes of the compositions were recorded. The particle size and polydispersity index (PDI) of HACH20 and Squalane@HACH (SQ@HACH) in the compositions (water solutions) were measured by dynamic light scattering (DLS, Malvern Zetasizer Nano ZS90, Malvern, UK). The HACH20 and Squalane@HACH (SQ@HACH) were stained by PTA negative staining and their morphology was observed by transmission electron microscopy (TEM, JEOL JEM-1400 electron microscopy, Tokyo, Japan).

Please refer to Table 3. As shown in Table 3, among the different oil@HACH particles, Paraffin oil@HACH particles were largest particles that approached around 1 μm after first homogenization by high shear mixer, presumably due to fluidity or viscosity characteristic of paraffin oil (an example of mineral oil). In contrast, other terpenoid oil@HACH particles were stable nanoscale particles suitable for injection. Furthermore, Squalene@HACH particles were smallest particles that approached around 190 nm after second homogenization by high pressure homogenizer. It is noted that particle size is highly correlated with the efficacy of immune stimulation, and terpenoid oil@HA-derivative particles may have better efficacy as an immune stimulator than mineral oil-forming particles. Additionally, Paraffin oil@HACH particles had significant phase separation after 24 hours storing at 4° C. refrigerator; It is demonstrated that Paraffin oil@HACH is not suitable for composing vaccines with antigens that require low temperature preservation.

TABLE 3
Characterization of different oil@HA-derivatives particles

| | First | Second | | Appearance after |
|---|---|---|---|---|
| Oil | Homogenization | Homogenization | Appearance | storing at 4° C. |
| Paraffin oil | Size: 956.5 ± 41.80 | Size: 452.0 ± 5.40 | Milk-like | Significant phase |
| | PDI: 0.660 ± 0.045 | PDI: 0.322 ± 0.029 | Suspension | separation |
| Squalane | Size: 488.3 ± 8.892 | Size: 372.4 ± 3.072 | Milk-like | Slight phase |
| | PDI: 0.289 ± 0.032 | PDI: 0.221 ± 0.013 | Suspension | separation |
| Squalene | Size: 310.6 ± 2.101 | Size: 190 ± 1.504 | Milk-like | Milk-like |
| | PDI: 0.141 ± 0.023 | PDI: 0.111 ± 0.010 | Suspension | suspension |
| Ocimene | Size: 577.5 ± 5.352 | Size: 592.9 ± 5.359 | Milk-like | Slight phase |
| | PDI: 0.380 ± 0.010 | PDI: 0.385 ± 0.025 | Suspension | separation |
| Farnesene | Size: 428.4 ± 10.3 | Size: 406.7 ± 2.538 | Milk-like | Milk-like |
| | PDI: 0.134 ± 0.025 | PDI: 0.210 ± 0.007 | Suspension | suspension |

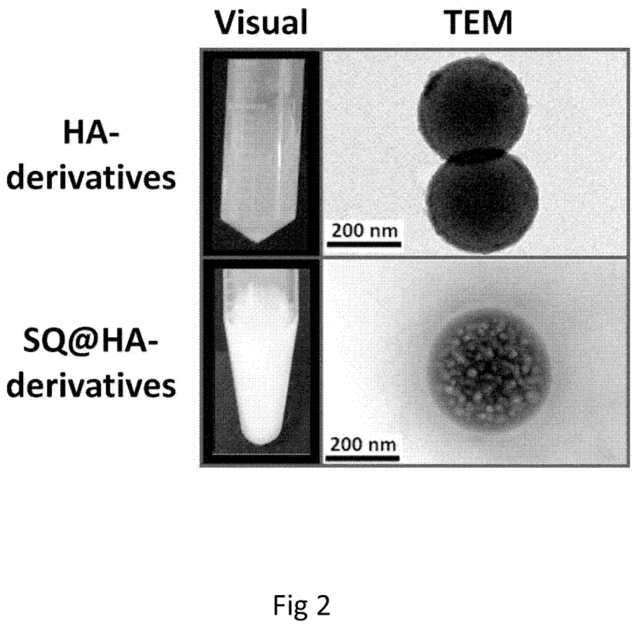

Please refer to FIG. 2. FIG. 2 is the Visual appearance and TEM images of HA-derivatives and SQ@HA-derivatives in the present invention.

As shown in FIG. 2, the resulting HA-derivatives product is cloudy and well dispersed in an aqueous solution. In the TEM image, we found that HA-derivatives could self-assemble to form particles that were approximately 200 nm.

In order to evaluate the potential of amphiphilic HA-derivatives as an emulsifier for stabilizing the oil/water interfaces, HA-derivatives were mixed with 5% squalene and then passed through a high-pressure homogenizer. As shown in FIG. 2, an isotropic emulsified formulation (named Squalane@HA-derivatives or SQ@HA-derivatives) was obtained after homogenization of the squalene/HA-derivatives/citrate buffer. The TEM images reveal that SQ@HA-derivatives spherical particles were composed of some squalene droplets (bright core) surrounded by the amphiphilic HA-derivatives (dark shell). The results of DLS of SQ@HA-derivatives show a uniform size of around 190±2 nm and a polydispersity of 0.136±0.027.

### Experiment 1-2 Analysis of the Oil Distribution in the Compositions Comprising Oil@HACH Particles

The compositions comprising Squalene@HACH particles were evenly divided into 2 equal portions. The first portion was mixed with an equal volume of ethyl acetate to make the squalene in the first portion (both of the squalene inside the Squalene@HACH particles and the squalene outside the Squalene@HACH particles) dissolve in the ethyl acetate phase. The ethyl acetate phase samples was collected and analyzed by GC-MS to determine the total content of squalene in the first portion.

The second portion was centrifuged to obtain the Squalene@HACH particles in the second portion. The obtained Squalene@HACH particles were mixed with an equal volume of ethyl acetate to make the squalene inside the Squalene@HACH particles dissolve in the ethyl acetate phase. The ethyl acetate phase samples was collected and analyzed by GC-MS to determine the total content of squalene inside the Squalene@HACH particles obtained from the second portion.

The proportion of the content of squalene inside the Squalene@HACH particles in the total content of squalene in the whole composition was calculated:

Proportion of the squalene inside the Squalene@HACH particles=total content of squalene inside the Squalene@HACH particles+total content of squalene in the composition

The results of this experiment show that at least 95% of squalene in the composition is encapsulated in the Squalene@HACH particles, based on the total content of squalene in the composition being 100% by mass.

### Experiment 1-3 Condition Suitable for Self-Assembly of Squalene@HACH Particles

In this experiment, the condition suitable for self-assembly of Squalene@HACH particles was assessed and shown in a Ternary diagram.

There were 28 groups in this experiment, that is, the first to the 28^{th} group. The preparation procedure of the compositions in each group was roughly the same as the experimental procedure of Composition comprising Squalene@HACH particles described in Experiment 1-1-4. The only differences were the weight of squalene, the weight of HACH, and the weight of sodium citrate solution (aqueous buffer solution).

Squalene@HACH particles in the compositions were detected and analyzed by dynamic light scattering, particle size analyzer, or transmission electron microscope. Moreover, please note that if the self-assembly of the Squalene@HACH particles occur in the composition, the composition would be a milk-like mixture solution (milk-like suspension). Conversely, if the self-assembly of the Squalene@HACH particles does not occur in the composition, phase separation of the oil and aqueous in the composition would occur (oil/water phase separation). In the case that the compositions comprise Squalene@HACH particles, the material ratio in the compositions would be used to create the shaded area in the Ternary diagram that shows the compositions able to be successfully prepared.

Please refer to Table 4. Table 4 is the result which shows the appearances of the 28 compositions comprising different amounts of of squalene, HACH, and aqueous buffer solution 28 groups in this experiment which shows the ratios of squalene, HACH, and aqueous buffer solution suitable for self-assembly of Squalene@HACH particles. The results show that squalene@HACH forms particles when the amount of squalene in the composition is less than 40% w/w (less than 40% w/w in HACH aqueous buffer solution). Furthermore, the inventors of the present invention found that the solubility of HACH was about 3% w/w in aqueous buffer. It is noted that 80 weight units of squalene can be coated by 1 weight unit of HACH by together forming squalene@HACH particles. For example, 40% w/w of squalene can be coated by 0.5% w/w of HACH by together forming squalene@HACH particles.

TABLE 4
Appearances of the compositions comprising different amounts
of squalene, HACH, and aqueous buffer solution

| | Aqueous buffer | HACH | Squalene | |
|---|---|---|---|---|
| Sample | (% w/w) | (% w/w) | (% w/w) | Appearance |
| #1 | 98.00 | 2.00 | 0 | ◯ |
| #2 | 93.34 | 1.90 | 4.76 | ◯ |
| #3 | 89.09 | 1.82 | 9.09 | ◯ |
| #4 | 81.66 | 1.67 | 16.67 | ◯ |
| #5 | 54.44 | 1.11 | 44.44 | X |
| #6 | 99.00 | 1.00 | 0 | ◯ |
| #7 | 94.29 | 0.95 | 4.76 | ◯ |
| #8 | 90.00 | 0.91 | 9.09 | ◯ |
| #9 | 82.50 | 0.83 | 16.67 | ◯ |
| #10 | 70.72 | 0.71 | 28.57 | ◯ |
| #11 | 55.00 | 0.56 | 44.44 | X |
| #12 | 94.00 | 1.00 | 5.00 | ◯ |
| #13 | 89.00 | 1.00 | 10.00 | ◯ |
| #14 | 84.00 | 1.00 | 15.00 | ◯ |
| #15 | 79.00 | 1.00 | 20.00 | ◯ |
| #16 | 74.00 | 1.00 | 25.00 | ◯ |
| #17 | 69.00 | 1.00 | 30.00 | ◯ |
| #18 | 64.00 | 1.00 | 35.00 | ◯ |
| #19 | 59.00 | 1.00 | 40.00 | ◯ |
| #20 | 54.00 | 1.00 | 45.00 | X |
| #21 | 49.00 | 1.00 | 50.00 | X |
| #22 | 79.50 | 0.50 | 20.00 | ◯ |
| #23 | 77.00 | 0.50 | 22.50 | ◯ |
| #24 | 74.50 | 0.50 | 25.00 | ◯ |
| #25 | 64.50 | 0.50 | 35.00 | ◯ |
| #26 | 62.00 | 0.50 | 37.50 | ◯ |
| #27 | 49.50 | 0.50 | 50.00 | X |
| #28 | 55.00 | 0.56 | 44.44 | X |

◯ means milk-like suspension; X means oil/water phase separation.

### Experiment 1-4 the Effects of Type of Oil@HACH on the Antigen-Presenting Cells (APC) Recruitment

There were 5 compositions in this experiment, comprising:
(A) composition comprising Paraffin oil@HACH particles (Paraffin oil@HACH emulsion);
(B) composition comprising Squalane@HACH particles (Squalane@HACH emulsion);
(C) composition comprising Squalene@HACH particles (Squalene@HACH emulsion);
(D) composition comprising Ocimene@HACH particles (Ocimene@HACH emulsion); and
(E) composition comprising Farnesene@HACH particles (Farnesene@HACH emulsion).

The preparation procedure of the compositions in this experiment was the same as the experimental procedure of compositions comprising oil@HACH particles described in Experiment 1-1-4.

15 BALB/c mice were randomly assigned into 5 groups (three mice per group), which were Paraffin oil@HACH group, Squalane@HACH group, Squalene@HACH group, Ocimene@HACH group, and Farnesene@HACH group.

50 μL of the composition comprising Paraffin oil@HACH particles, composition comprising Squalane@HACH particles, composition comprising Squalene@HACH particles, composition comprising Ocimene@HACH particles, or composition comprising Farnesene@HACH particles (the test samples in this experiment) was injected intramuscularly into the right quadriceps of mice in the Paraffin oil@HACH group, Squalane@HACH group, Squalene@HACH group, Ocimene@HACH group, and Farnesene@HACH group, respectively; and 50 μL of sodium citrate buffer (pH=6.5) (the vehicle control in this experiment) was injected intramuscularly into the left quadriceps of mice in these groups to monitor the effect induced by the injection on each mouse.

The mice in each group were sacrificed at 5^{th} day after injection.

The left quadriceps muscles and the right quadriceps muscles of each mouse were harvested respectively. The tendons were removed, and muscles were digested with 3 mL of phosphate buffered saline (PBS) containing 0.05% (w/v) type II collagenase, 10 μg/mL of DNase I, and 0.5% BSA at 37° C. for 40 min. The muscle digestion was quenched by the addition of excess medium; and then the cell suspension was collected by centrifugation at 1200 rpm for 5 min, resuspended in PBS, and filtered through a 70-μm nylon mesh (BD-Biosciences).

The cell suspensions obtained from the left quadriceps muscles and the right quadriceps muscles of each mouse were respectively mixed with a medium containing titrated fluorescent conjugates of monoclonal antibodies CD11b-FITC (BD Pharmingen) and CD11c-PE (Thermo Fisher). The cells were incubated at 4° C. in a dark environment for 40 min. Finally, the stained cells were washed with staining buffer and analyzed by BD Accuri™ C6 flow cytometer.

The degree of dendritic cell (CD11c⁺) recruitment in each mouse was assessed according to the following formula (VI):

$$degree of dendritic cell ( CD 11 {c}^{+} ) recruitment = the total CD 11 {c}^{+} cells in the right quadriceps muscles \div the total CD 11 {c}^{+} cells in the left quadriceps muscles .$$

The degree of macrophage cell (CD11b⁺) recruitment in each mouse was assessed according to the following formula (VII):

$$degree of macrophage cell ( CD 11 {b}^{+} ) recruitment = the total CD 11 {b}^{+} cells in the right quadriceps muscles \div the total CD 11 {b}^{+} cells in the left quadriceps muscles .$$

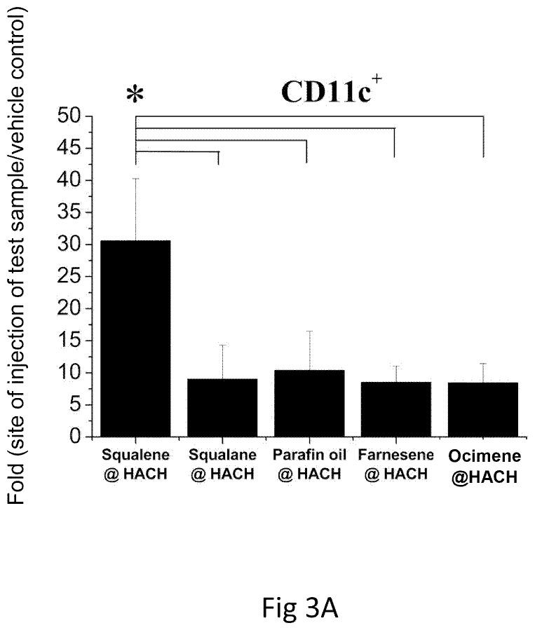

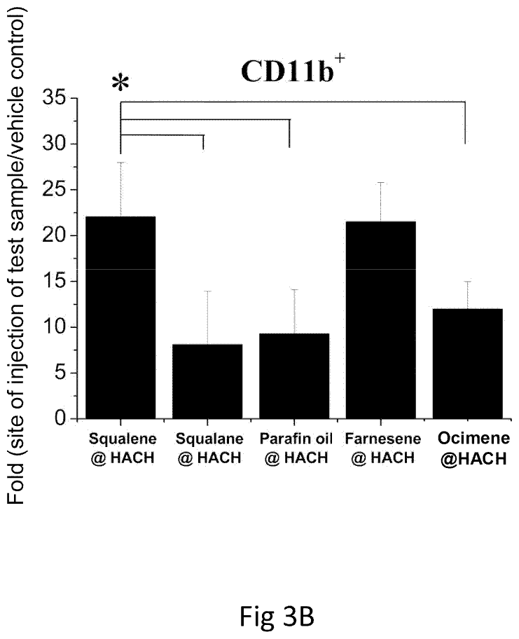

Please refer to FIGS. 3A and 3B. FIG. 3A is result which shows the effect of oil@HACH on dendritic cells recruitment. FIG. 3B is result which shows the effect of oil@HACH on macrophages recruitment. Please note that the symbol “*” represents the group is significant different with the Squalene@HACH group.

As shown in FIG. 3A, these compositions comprising oil@HACH particles could recruit dendritic cells (CD11c⁺), wherein the composition comprising Squalene@HACH particles recruits more dendritic cells (CD11c⁺) than the compositions comprising other oil@HACH particles. As shown in FIG. 3B, these compositions comprising oil@HACH particles could recruit macrophages (CD11b⁺), wherein the composition comprising Squalene@HACH particles or Farnesene@HACH particles recruits more macrophages (CD1 b⁺) than the compositions comprising other oil@HACH particles.

These results suggested that for the use in the stimulation of immune response, oils classed as unsaturated terpenoid (such as Squalene), terpenoid consisting of equal to or less than 3 units of terpene carbon skeleton (or equal to or less than 6 units of isoprene carbon skeletons such as squalane, squalene, ocimene, and farnesene), and/or paraffin oil are suitable for preparing oil@HACH particles. Preferably, for the use in the stimulation of immune response, oils classed as unsaturated terpenoid (such as Squalene) and/or terpenoid consisting of less than 3 units of terpene carbon skeleton (or less than 6 units of isoprene carbon skeleton such as ocimene and farnesene) are more suitable for preparing oil@HACH particles.

### Experiment 1-5 the Effects of the Reactants in the Oil@HACH Synthesis Process on the Antigen-Presenting Cells (APC) Recruitment

12 BALB/c mice were randomly assigned into 4 groups (three mice per group), which were cholesterol group, squalene group, hyaluronic acid group, and Squalene@HACH group.

The experimental procedure was roughly the same as that described in Experiment 1-4. The only differences were the compositions administered to the mice.

Cholesterol group: 50 μL of the N-methyl-2-pyrrolidone (NMP) solution comprising cholesterol (0.25 mg/mL) (the test sample in this group) was injected intramuscularly into the right quadriceps of mice; and 50 μL of N-methyl-2-pyrrolidone solution (the vehicle control in this group) was injected intramuscularly into the left quadriceps of mice to monitor the effect induced by the injection on each mouse.

Squalene group: 50 μL of the N-methyl-2-pyrrolidone (NMP) solution comprising squalene (50 μL/mL) (the test sample in this group) was injected intramuscularly into the right quadriceps of mice; and 50 μL of N-methyl-2-pyrrolidone solution (the vehicle control in this group) was injected intramuscularly into the left quadriceps of mice to monitor the effect induced by the injection on each mouse.

Hyaluronic acid group: 50 μL of the sodium citrate solution comprising Hyaluronic acid (8.73 mg/mL) and Glycine (0.25 mg/mL) (the test sample in this group) was injected intramuscularly into the right quadriceps of mice; and 50 μL of sodium citrate buffer (pH=6.5) (the vehicle control in this group) was injected intramuscularly into the left quadriceps of mice to monitor the effect induced by the injection on each mouse.

Squalene@HACH group: 50 μL of the composition comprising Squalene@HACH particles (cholesterol content: 0.0589 mg; squalene content: 2.5 μL; hyaluronic acid content: 0.4325 mg) prepared by the method as described in Experiment 1-1-4 (the test sample in this group) was injected intramuscularly into the right quadriceps of mice; and 50 μL of sodium citrate buffer (pH=6.5) (the vehicle control in this group) was injected intramuscularly into the left quadriceps of mice in these groups to monitor the effect induced by the injection on each mouse.

The mice in each group were sacrificed at 5^{th} day after injection.

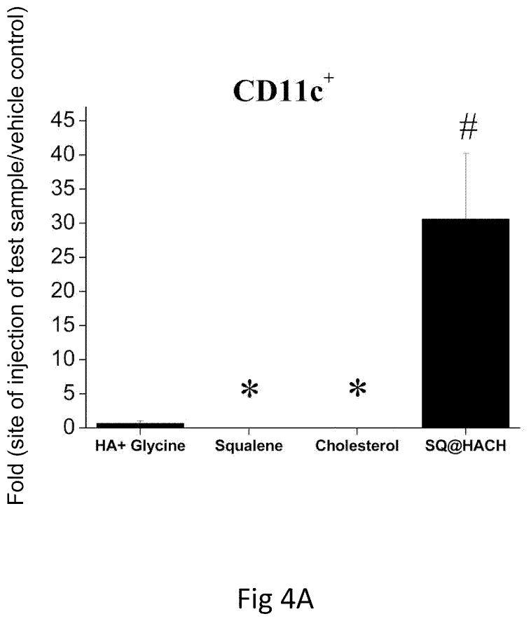

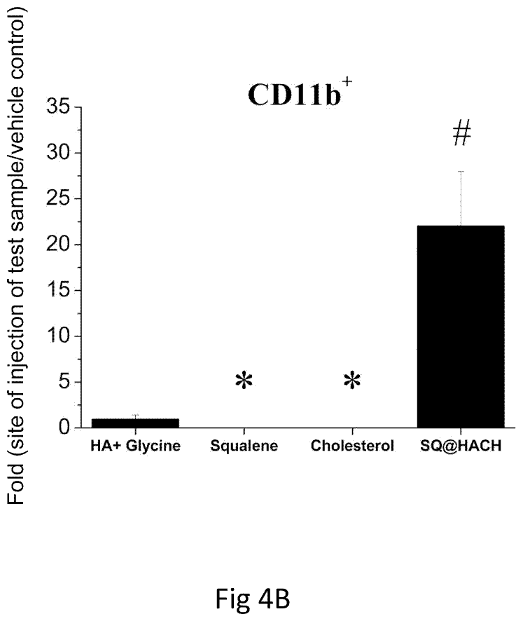

Please refer to FIGS. 4A and 4B. FIG. 4A is result which shows the effect of reactants and product in the Squalene@HACH synthesis process on dendritic cells recruitment. FIG. 4B is result which shows the effect of reactants and product in the Squalene@HACH synthesis process on macrophages recruitment. Please note that the symbol “*” represents the animals in this group died during the experiment and “#” means the group has significant difference compared with other three groups.

As shown in FIGS. 4A and 4B, all of the reactants in the Squalene@HACH synthesis process (cholesterol, squalene, and hyaluronic acid) lack the ability to recruit dendritic cells (CD11c+) and macrophages (CD11b+). Unexpectedly, Squalene@HACH particles could recruit more than 35 folds of dendritic cells (CD11c⁺) and more than 25 folds of macrophages (CD11b⁺).

As shown in FIG. 4A, comparison among the dendritic cells recruitment efficacy of the cholesterol group, squalene group, hyaluronic acid group, and Squalene@HACH group (SQ@HACH group) demonstrated that cholesterol, squalene, and hyaluronic acid in the Squalene@HACH particles manifests synergy in the dendritic cells recruitment efficacy.

As shown in FIG. 4B, comparison among the macrophages recruitment efficacy of the cholesterol group, squalene group, hyaluronic acid group, and Squalene@HACH group (SQ@HACH group) demonstrated that cholesterol, squalene, and hyaluronic acid in the Squalene@HACH particles manifests synergy in the macrophages recruitment efficacy.

### Experiment 1-6 the Effects of the Mixture Comprising Squalene@HACH and Targeted Antigen on the Antigen-Presenting Cells (APC) Recruitment

36 BALB/c mice were randomly assigned into 3 groups (twelve mice per group), which were WT1 group, WT1+Squalene@HACH group, and WT1+Addvax group. 12 mice in each group were randomly assigned into four sub-groups (three mice per sub-group) for the different administration periods (administered for 1, 3, 5, or 7 days).

The composition comprising Squalene@HACH particles (Squalene@HACH emulsion) were prepared by the method as described in Experiment 1-1-4. The experimental procedure was roughly the same as that described in Experiment 1-4. The only differences were the compositions administered to the mice.

WT1 group: 50 μL of the sodium citrate solution comprising 25 μL of WT1 protein (the test sample in this group) was injected intramuscularly into the right quadriceps of 12 mice; and 50 μL of sodium citrate buffer (pH=6.5) (the vehicle control in this group) was injected intramuscularly into the left quadriceps of 12 mice to monitor the effect induced by the injection on each mouse.

WT1+Squalene@HACH group: 50 μL of the composition comprising 25 μL of WT1 protein and 25 μL of the composition comprising Squalene@HACH particles (Squalene@HACH emulsion) (the test sample in this group) was injected intramuscularly into the right quadriceps of 12 mice; and 50 μL of sodium citrate buffer (pH=6.5) (the vehicle control in this group) was injected intramuscularly into the left quadriceps of 12 mice to monitor the effect induced by the injection on each mouse.

WT1+Addvax group: 50 μL of the composition comprising 25 μL of WT1 protein and 25 μL of Addvax™ (a MF59 like squalene adjuvant for vaccine research, purchased from Invivo gen, San Diego, LA, USA) (the test sample in this group) was injected intramuscularly into the right quadriceps of 12 mice; and 50 μL of sodium citrate buffer (pH=6.5) (the vehicle control in this group) was injected intramuscularly into the left quadriceps of 12 mice to monitor the effect induced by the injection on each mouse.

The mice in each group were sacrificed at 1^{st}, 3^{rd}, 5^{th}, or 7^{th} day after injection.

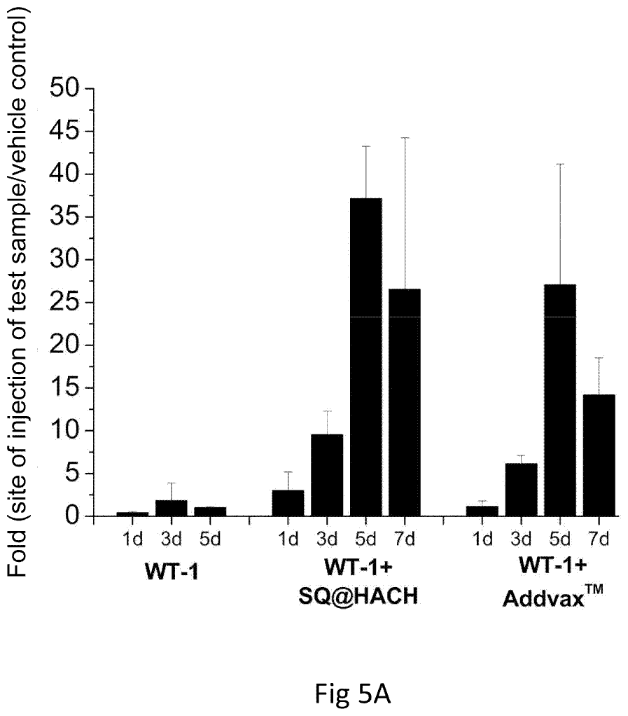

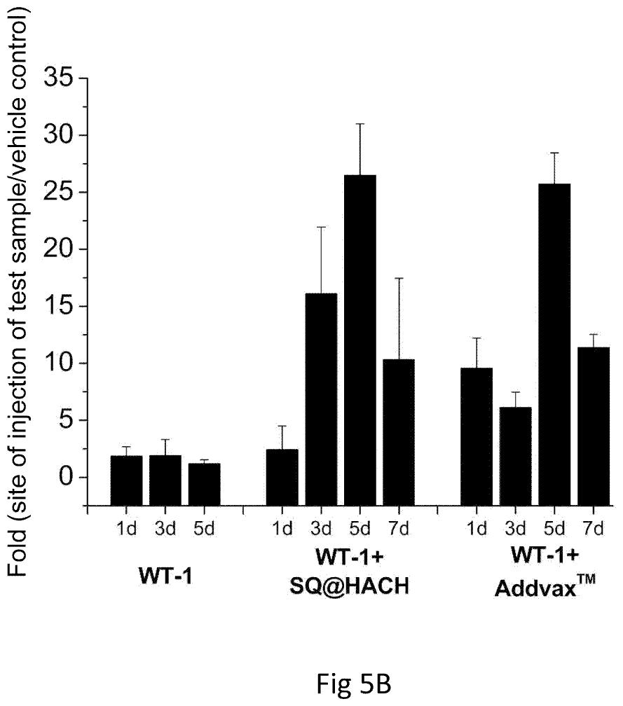

Please refer to FIGS. 5A and 5B. FIG. 5A is result which shows the effect of the mixture comprising Squalene@HACH and targeted antigen on dendritic cells recruitment. FIG. 5B is result which shows the effect of the mixture comprising Squalene@HACH and targeted antigen on macrophages recruitment.

As shown in FIGS. 5A and 5B, WT1 alone lacks the ability to recruit dendritic cells (CD11c+) and macrophages (CD11b+). Unexpectedly, the mixture comprising WT1 and Squalene@HACH (WT1+Squalene@HACH group, also known as WT-1+SQ@HACH group herein) could recruit more than 35 folds of dendritic cells (CD11c⁺) and more than 25 folds of macrophages (CD11b⁺) at 5^{th} day after injection.

Therefore, the oil@HACH particles in the present invention could stimulate an immune response such as recruiting dendritic cells and/or recruiting macrophages, and therefore oil@HACH particles could be mixed with active ingredient or targeted antigen (or antigen) for treating or preventing a disease or disorder. Moreover, the oil@HACH particles in the present invention could stimulate an immune response and therefore is an efficient adjuvant suitable for vaccine use (such as for cancer vaccine use, or other disease or disorder vaccine use).

### Experiment 1-7 the Effects of the Mixture Comprising Squalene@HACH and Targeted Antigen on T Cell Immunity

12 BALB/c mice were randomly assigned into 4 groups (three mice per group), which were PBS group, WT1 group, WT1+IFN-alpha-based adjuvant group, and WT1+Squalene@HACH group.

The Squalene@HACH particles were prepared by the method as described in Experiment 1-1-4.

PBS group: 50 μL of the phosphate buffered saline was injected intramuscularly into both of the left and right quadriceps of mice on day 0 and day 14.

WT1 group: 50 μL of the sodium citrate solution comprising 25 μL of WT1 protein was injected intramuscularly into both of the left and right quadriceps of mice on day 0 and day 14.

WT1+IFN-alpha-based adjuvant group: 50 μL of the sodium citrate solution comprising 25 μL of WT1 protein and 25 μL of IFN-alpha-based adjuvant (supplied from Latham Medical Institution, Japan; the IFN-alpha-based adjuvant was used as a positive control) was injected intramuscularly into both of the left and right quadriceps of mice on day 0 and day 14.

WT1+Squalene@HACH group: 50 μL of the sodium citrate solution comprising 25 μL of WT1 protein and 25 μL of the composition comprising Squalene@HACH particles (Squalene@HACH emulsion) was injected intramuscularly into both of the left and right quadriceps of mice on day 0 and day 14.

The mice in each group were sacrificed on day 28.

The spleens were harvested and temporarily pooled in a sterile lymphocyte culture medium (LCM) to avoid cell death, wherein the lymphocyte culture medium contained 900 mL of RPMI 1640, 100 mL of FBS, 25 mL of HEPES (25 mM), and 50 μL of P-mercaptoethanol (150 M).

The splenocytes were isolated by the following procedure: the spleen was gently pressed with the plunger seal of a 5-mL syringe on a 70-μm cell strainer into a 50-mL tube and washed through a cell strainer with RPMI 1640 containing 10% (v/v) FBS. Then the washing buffer was removed by centrifugation at 1200 rpm for 5 mins at 4° C. The cell pellet was resuspended in 5 mL Red Blood Cell lysis buffer (dilution from RBC lysis buffer (10×), BioLegend, lot: B166991) and incubated in an ice bath. Then, 30 mL of cold PBS was added for lysis quenching, and the supernatant was removed by centrifugation at 1,200 rpm for 5 min at 4° C. Finally, the cell pellets were gently resuspended in LCM. The cell solution was counted and diluted to the desired cell concentrations.

The cell suspensions obtained from the spleen of mice in each group were respectively mixed with a medium containing titrated fluorescent conjugates of monoclonal antibodies CD8a-Alexa Fluor 647 (BD Pharmingen), and CD4-FITC (Thermo Fisher). The cells were incubated at 4° C. in a dark environment for 40 min. Finally, the stained cells were washed with staining buffer and analyzed by BD Accuri™ C6 flow cytometer.

The results show that there was the highest amount of T cells in the WT-1+Squalene@HACH group (CD4⁺ T cell: 37.9%; CD8⁺ T cell: 33.0%), which was higher than the amount of T cells in the PBS group (CD4⁺: 29.2%; CD8⁺: 24.7%), WT-1 group (CD4⁺: 34.1%; CD8⁺: 27.5%), and WT1+IFN-alpha-based adjuvant group (CD4⁺: 33.0%; CD8⁺: 31.7%). These results show that the T cell immunity induced by Squalene@HACH particles was higher than the T cell immunity induced by the common clinical adjuvant IFN-alpha.

Therefore, the oil@HACH particles successfully stimulating an immune response such as inducing T cell immunity and therefore could be mixed with active ingredient or targeted antigen (or antigen, or antigenic component) for treating or preventing a disease or disorder. Moreover, the oil@HACH particles in the present invention could stimulate an immune response, and therefore is an efficient adjuvant suitable for vaccine use (such as for cancer vaccine use, or other disease or disorder vaccine use).

### Experiment 1-8 the Effects of the Mixture Comprising Squalene@HACH and Targeted Antigen on Antigen-Specific T Cell Response

The splenocytes obtained from the spleen of mice in the WT1 group, WT1+IFN-alpha-based adjuvant group, and WT1+Squalene@HACH group described in Experiment 1-7 were respectively used in the WT1 group, WT1+IFN-alpha-based adjuvant group, and WT1+Squalene@HACH group in this experiment.

The splenocytes obtained from the spleen of mice in the same group described in Experiment 1-7 were pooled together. 2×10⁶ splenocytes in each group were suspended in 1 mL of LCM and added to 24-well plates. The splenocytes in each group was re-stimulated with 10 μL of WT1 antigen at 37° C. for 72 hours. The supernatant was collected for multiplex cytokine assays (IL-2, IL-4, IL-6, IL-10, IL-17, IFN-γ, and TNF-α) by using the cytometric bead array (CBA) mouse Th1/Th2/Th17 cytokine kit according to the manufacturer's instructions.

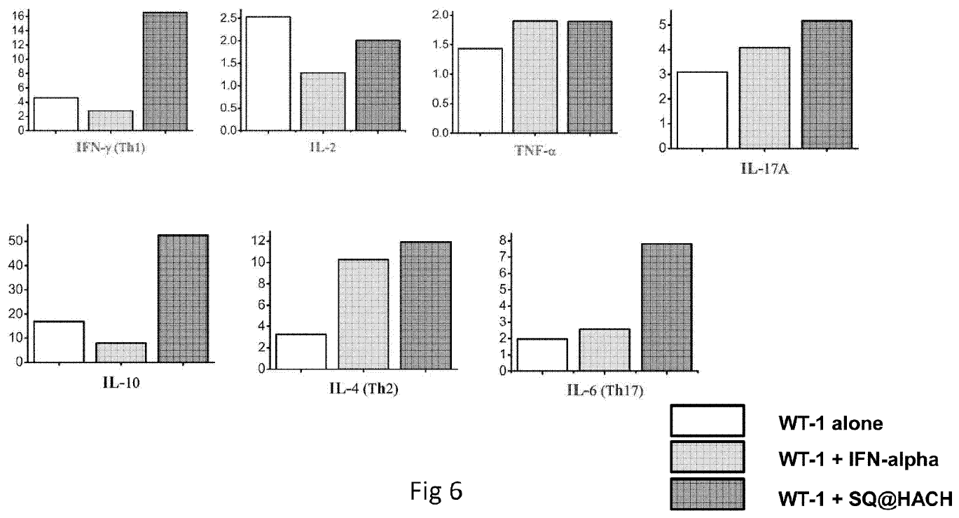

Please refer to FIG. 6. FIG. 6 is the result which shows the effect of the mixture comprising Squalene@HACH and targeted antigen on T cell-related cytokine production.

As shown in FIG. 6, there was the highest amount of T cell-related cytokines (IL-2, IL-4, IL-6, IL-10, IL-17, IFN-γ, and TNF-α) produced in the WT1+Squalene@HACH group (WT-1+SQ@HACH group), which was higher than the amount of T cell-related cytokines produced in the WT-1 group and in the WT1+IFN-alpha-based adjuvant group. These results show that the antigen-specific T cell immunity induced by Squalene@HACH particles was higher than the antigen-specific T cell immunity induced by the common clinical adjuvant IFN-alpha.

These results indicate that oil@HACH particles successfully stimulate antigen-specific T-cell immunity and induce the production of the Th1/Th2/Th17-related cytokines after antigen restimulation.

Therefore, the oil@HACH particles successfully stimulating an immune response such as inducing antigen-specific T cell immunity and Th1/Th2/Th17-related cytokines production.

That is, the oil@HACH particles in the present invention could be mixed with active ingredient or targeted antigen (or antigen, or antigenic component) for treating or preventing a disease or disorder. Moreover, the oil@HACH particles in the present invention could stimulate an immune response, and therefore are an efficient adjuvant suitable for vaccine use (such as for cancer vaccine use, or other disease or disorder vaccine use).

### Experiment 1-9 the Anti-Tumor Activity of the Mixture Comprising Oil@HACH and Targeted Antigen

### Experiment 1-9-1 Ability of the Mixture Comprising Oil@HACH and Targeted Antigen to Treat and Prevent Breast Cancer

18 BALB/c mice were randomly assigned into 3 groups (six mice per group), which were PBS group, WT1 group, and WT1+Squalene@HACH group.

The composition comprising Squalene@HACH particles was prepared by the method as described in Experiment 1-1-4.

PBS group: 50 μL of the phosphate buffered saline was injected intramuscularly into both of the left and right quadriceps of mice on Day −14 and Day 0.

WT1 group: 50 μL of the sodium citrate solution comprising 25 μL of WT1 protein was injected intramuscularly into both of the left and right quadriceps of mice on Day −14 and Day 0.

WT1+Squalene@HACH group: 50 μL of the sodium citrate solution comprising 25 μL of WT1 protein and 25 μL of the composition comprising Squalene@HACH particles was injected intramuscularly into both of the left and right quadriceps of mice on Day −14 and Day 0.

On Day 0, after the injection process as described above was completed, 0.05 mL of the phosphate buffered saline (PBS) comprising 4T1 cells (2×10⁶ cells/mL; 4T1 cell is abreast cancer cell line) was injected subcutaneously into the abdominal mammary fat pad of the mice in each group, and the tumor volume and survival status of mice were initially monitored on this day (Day 0). Tumor volume was calculated by ½*(4*3.14/3)*(length/2)*(width/2)*height*1000, and the observation of the tumor volume and survival status was stopped if the mouse was defined as either death or paralysis or the tumor volume of the mouse exceeding 1000 mm³.

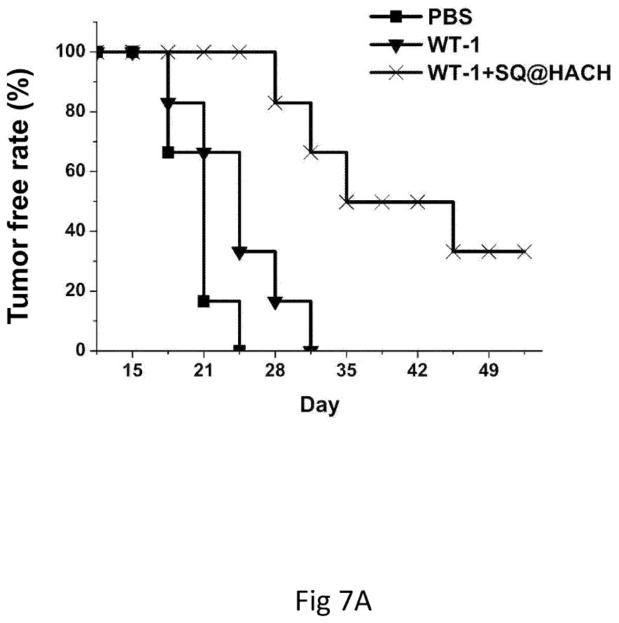

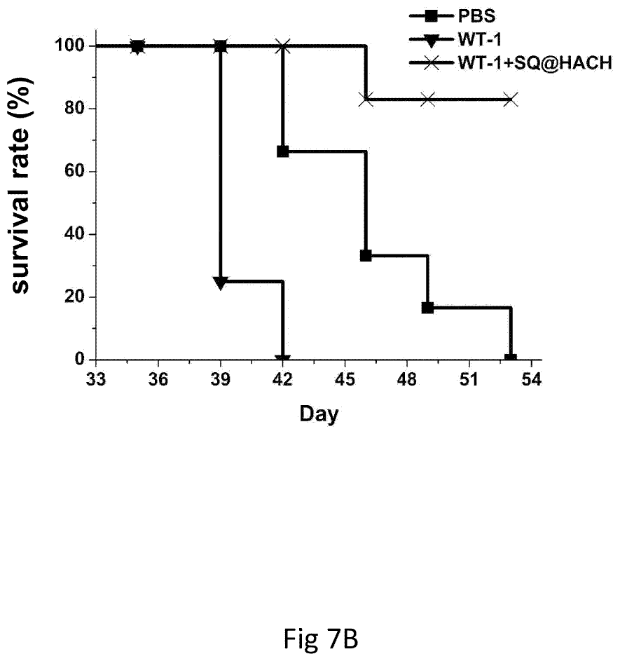

Please refer to Table 5 and FIGS. 7A and 7B, which provide results from mouse model studies demonstrating the anti-tumor activities of WT1+Squalene@HACH.

As shown in Table 5, tumor developed in all of the 6 mice of the PBS group (tumor was initially developed between Day 18-32); similarly, tumor developed in all of the 6 mice of the WT1 group (tumor was initially developed between Day 18-25). Unexpectedly, for 4 mice in the WT1+Squalene@HACH group, a significant delay in the development of tumor was observed (tumor was initially developed between Day 25-46); and more surprisingly, no tumor developed in the remaining 2 mice in this WT1+Squalene@HACH group (WT1+SQ@HACH group). These results demonstrated that oil@HACH particles in the present invention could successfully prevent tumor formation and prevent tumor development.

Moreover, as shown in Table 5, all of the 6 mice in the PBS group died on Day 53; similarly, all of the 6 mice in the WT group died on Day 53. Unexpectedly, 5 mice in the WT1+Squalene@HACH group were still alive on Day 53 (only 1 mouse in this WT1+Squalene@HACH group died on Day 53, but this mouse was died from “attack by other mice” instead of “the tumor”). These results demonstrated that oil@HACH particles in the present invention could successfully prolong the life of a subject with cancer or tumor disease.

TABLE 5
The results from mouse model studies demonstrating
the anti-tumor activities of WT1 with SQ@HACH

| | | | | The day of | Survival |
|---|---|---|---|---|---|
| | Animal | Activation | | the Tumor | Status on |
| Group | Number | ingredient | Adjuvant | Growth | Day 53 |
| PBS | 1 | — | — | Day 18 | Dead |
| | 2 | — | — | Day 21 | Dead |
| | 3 | — | — | Day 25 | Dead |
| | 4 | — | — | Day 25 | Dead |
| | 5 | — | — | Day 28 | Dead |
| | 6 | | | Day 32 | Dead |
| WT1 | 7 | WT 1 | — | Day 18 | Dead |
| | 8 | WT 1 | | Day 21 | Dead |
| | 9 | WT 1 | — | Day 21 | Dead |
| | 10 | WT 1 | — | Day 25 | Dead |
| | 11 | WT 1 | — | Day 25 | Dead |
| | 12 | WT 1 | — | Day 25 | Dead |
| WT1 + | 13 | WT 1 | SQ@HACH | Day 25 | Dead * |
| SQ@HACH | 14 | WT 1 | SQ@HACH | Day 32 | Survival |
| | 15 | WT 1 | SQ@HACH | Day 35 | Survival |
| | 16 | WT 1 | SQ@HACH | Day 46 | Survival |
| | 17 | WT 1 | SQ@HACH | ND | Survival |
| | 18 | WT 1 | SQ@HACH | ND | Survival |

Dead means that the mouse died because of the tumor;
Dead * means that the mouse died from attack by other mice
ND means that the mouse did not grow tumor

Therefore, the oil@HACH particles mixed with active ingredient or targeted antigen (or antigen) can successfully stimulate an immune response to prevent or treat diseases or disorders such as cancer or tumor. Moreover, the oil@HACH particles in the present invention could stimulate an immune response and therefore are an efficient adjuvant suitable for vaccine use (such as for cancer vaccine use, or other disease or disorder vaccine use).

### Experiment 1-9-2 Ability of the Mixture Comprising Oil@HACH and Antigen to Treat and Prevent Lymphoma

9 BALB/c mice were randomly assigned into 3 groups (three mice per group), which were PBS group, OVA group, and OVA+Squalene@HACH group.

The composition comprising Squalene@HACH particles was prepared by the method as described in Experiment 1-1-4.

PBS group: 50 μL of the phosphate buffered saline was injected intramuscularly into both of the left and right quadriceps of mice on Day −14 and Day 0.

OVA group: 50 μL of the sodium citrate solution comprising 25 μg of OVA protein (ovalbumin, a well known antigen commonly be used as a model antigen in vaccine formulation) was injected intramuscularly into both of the left and right quadriceps of mice on Day −14 and Day 0.

WT1+Squalene@HACH group: 50 μL of the sodium citrate solution comprising 20 g of OVA protein and 25 μL of the composition comprising Squalene@HACH particles was injected intramuscularly into both of the left and right quadriceps of mice on Day −14 and Day 0.

On Day 0, after the injection process as described above was completed, 0.1 mL of the phosphate buffered saline (PBS) comprising E.G7-OVA cells (3×10⁵ cells/mL; E.G7-OVA cell is a lymphoma cell line) was injected subcutaneously into the abdominal mammary fat pad of the mice in each group, and the tumor volume and survival status of mice were initially monitored on this day (Day 0). Tumor volume was calculated by ½*(4*3.14/3)*(length/2)*(width/2)*height*1000, and the observation of the tumor volume and survival status was stopped if the mouse was defined as either death or paralysis or the tumor volume of the mouse exceeding 1500 mm³.

Inventors of the present invention found that oil@HACH particles in the present invention could successfully prevent tumor formation and prevent tumor development as well as prolong the life of a subject with cancer or tumor disease.

### Experiment 1-10 Ability of the Mixture Comprising Oil@HACH and Targeted Antigen to Treat and Prevent Infectious Disease

9 BALB/c mice were randomly assigned into 3 groups (three mice per group), which were PBS group, split-H7N9 group, and split-H7N9+Squalene@HACH group.

The composition comprising Squalene@HACH particles was prepared by the method as described in Experiment 1-1-4.

PBS group: 50 μL of the phosphate buffered saline was injected intramuscularly into both of the left and right quadriceps of mice on Day −28 and Day −14.

Split-H7N9 group: 50 μL of the sodium citrate solution comprising 0.5 μg of split-H7N9 protein (an influenza H7N9 vaccine antigen, purchased from ADIMMUNE Corporation, Taiwan, China) was injected intramuscularly into both of the left and right quadriceps of mice on Day −28 and Day −14.

Split-H7N9+Squalene@HACH group: 50 μL of the sodium citrate solution comprising 0.5 μg of split-H7N9 protein and 25 μL of the composition comprising Squalene@HACH particles was injected intramuscularly into both of the left and right quadriceps of mice on Day −28 and Day −14.

On Day 0, serum samples from immunized mice in each group were collected by withdrawing submandibular blood at predetermined times, and they underwent centrifugation at 7500 rpm for 15 min.

The hemagglutination inhibition (HI) test for influenza virus was used in this experiment to quantify the relative concentration of influenza viruses. Actual analyzed methods are known, or will be apparent, to those skilled in the art (De et al., 2003). In the hemagglutination inhibition (HI) test described here, series dilutions of serum from immunized mice are incubated with a virus (an influenza H7N9 vaccine antigen, supplied from ADIMMUNE Corporation, Taiwan, China), and erythrocytes are added. After incubation, the HI titer is read as the highest dilution of serum that inhibits hemagglutination.

The preliminary data of this experiment suggested that the composition comprising split-H7N9 and Squalene@HACH particles could induce a geometric mean titer (GMT) of 127 at week 2, which means that oil@@HACH particles in this invention could induce sufficient neutralizing antibodies to prevent H7N9 virus infection.

Therefore, the oil@HACH particles mixed with active ingredient or targeted antigen (or antigen) in this invention could be used to stimulate an immune response to prevent or treat diseases or disorders such as infectious disease (viral infection, fungal infection, protozoan infection, or bacterial infection). Moreover, the oil@HACH particles in the present invention could stimulate an immune response, and therefore are an efficient adjuvant suitable for vaccine use (such as for cancer vaccine use, infectious disease vaccine use, or other disease or disorder vaccine use).

### Experiment 2: Composition Comprising Oil@HAODA Particles

### Experiment 2-1 Preparation of Composition Comprising Oil@HAODA Particles

In this embodiment, hyaluronic acid-octadecylamine conjugates (HAODA), which is a macromolecule of the present invention, was used.

Equation (e) provides the synthetic pathways of hyaluronic acid-octadecylamine conjugates (HAODA) of the present invention.

Equation (e) Synthesis route of HAODA (Conjugation of HA and octadecylamine):

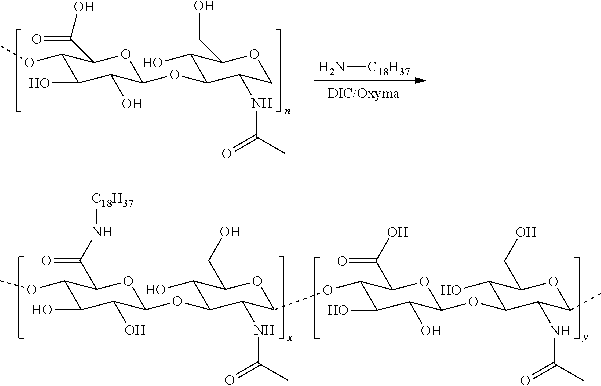

And wherein n, x, and y each individually represent an integer value, n=x+y, x≥1, y≥x, and x+y≥3, such as x+y≥10.

### Experiment 2-1-1 Synthesis Route of HAODA (Conjugation of HA and Octadecylamine)

Please refer to equation (e), in this step for the preparation of HAODA, octadecylamine was conjugated to the carboxylic group of HA by DIC/Oxyma activation to form the HAODA. The detailed process in this final step comprises:

200 mg (0.5 mmol, 1.0 eq.) of hyaluronic acid (36 kDa or 360 kDa) was first dissolved in a mixture of 20 mL of water and 80 mL of acetone. A mixture containing 50 mg (0.55 mmol, 1.1 eq.) of Oxyma and 20 mg (0.15 eq.) of octadecylamine was dissolved in 32 mL of acetone and then the resulting solution was added into the hyaluronic acid solution (HA solution). The mixed solution was slowly added by 162 μL (1.0 mmol, 2.0 eq.) of DIC and stirred for 24 hours. The obtained solution was transferred into a 3500-MWCO dialysis bag and purified by sequential dialysis against acetone/water (50/50, v/v), 0.3 M NaCl aqueous solution, and pure water. Finally, water was removed from the dialyzed product solution by freeze-drying to obtain HAODA15. The different degree of substitution (DS) of HAODA was synthesized by adding corresponding equivalents of octadecylamine; HAODA5, HAODA15, and HAODA25 means 0.05, 0.15 and 0.25 equivalent of octadecylamine for the HA conjugation, respectively. The conjugation ratio (DS %) of HAODA was shown in Table 6 and determined by ¹H-NMR spectroscopy (Agilent Technologies 400 MHz NMR).

The DS of HAODA was analyzed by an ¹H-NMR spectroscopy and calculated using the following formula (VIII):

$$DS = \frac{{A}_{ODA}}{{A}_{HA}} \times 100 \%$$

where A_{ODA} and A_{HA} respectively represent the integral area of terminal methyl group of octadecylamine (δ 0.8-0.9, broad s, 3H), and the integral area of N-acetyl group at glucosamine of HA (δ 1.8-2.2, broad s, 3H). Actual analyzed methods are known, or will be apparent, to those skilled in the art.

Please refer to Table 6. The DS % of different molecular weight (MW) HAODA and their estimated DS ratios were listed. The DS % of 36k-HAODA5, 36k-HAODA15, and 36k-HAODA25 were 5.21%, 15.61%, and 23.36%, respectively. The DS % of 360k-HAODA5, 360k-HAODA15, and 360k-HAODA25 (which were the HAODAs with higher MW) were 5.01, 15.42, and 26.52, respectively. These DS ratios were closed to correspondingly estimated DS ratios.

TABLE 6
Calculation of DS % of HAODA by ¹H-NMR spectroscopy

| | DS(%) |
|---|---|
| HA | — |
| 36k-HAODA5 | 5.21 |
| 36k-HAODA15 | 15.61 |
| 36k-HAODA25 | 23.36 |
| 360k-HAODA5 | 5.01 |
| 360k-HAODA15 | 15.42 |
| 360k-HAODA25 | 26.52 |

Moreover, the hydrophilic-lipophilic balance value (HLB value) of amphiphilic HA-derivatives was calculated according to the Griffin's method and expressed as the above described formula (V).

Please refer to Table 7. As shown in Table 7, the HLB value of the HAODAs in this experiment ranges from 14 to 20.

TABLE 7
Calculation of HLB value of amphiphilic HA-derivatives

| | DS(%) | HLB value |
|---|---|---|
| HA | 0 | 20.0 |
| 36k-HAODA5 | 5.21 | 19.0 |
| 36k-HAODA15 | 15.61 | 16.9 |
| 36k-HAODA25 | 23.36 | 15.3 |
| 360k-HAODA5 | 5.01 | 19.0 |
| 360k-HAODA15 | 15.42 | 16.9 |
| 360k-HAODA25 | 26.52 | 14.7 |

### Experiment 2-1-2 Preparation of the Composition Comprising Oil@HAODA Particles

In this experiment, different kinds of oils were used to prepare compositions comprising oil@HAODA particles. These oils were:
(1) paraffin oil, which is a mineral oil.
(2) squalane, which is a saturated terpenoid consisting of 3 terpenes (triterpene) and is classified as a saturated triterpene.
(3) squalene, which is an unsaturated terpenoid consisting of 3 terpenes (triterpene) and is classified as an unsaturated triterpene.
(4) ocimene, which is a terpenoid consisting of 1 terpene (monoterpene) and is classified as a monoterpene.
(5) farnesene, which is a terpenoid consisting of 1.5 terpenes (sesquitterpene) and is classified as a sesquiterpene.

100 mg of HAODA (the final concentration in the composition is 1% (w/v)) was dissolved in 9.5 mL of sodium citrate solution (pH=6.5, 10 mM), and then 500 μL of oil (the final concentration in the composition is 5% (v/v)) was added into the HAODA solution. The resulting solution was pre-mixed in high shear mixer for 10 mins and then homogenized through a high-pressure homogenizer to obtain compositions comprising different oil@HACH particles including:
(A) Composition comprising Paraffin oil@HAODA particles (Paraffin oil@HAODA emulsion);
(B) Composition comprising Squalane@HAODA particles (Squalane@HAODA emulsion);
(C) Composition comprising Squalene@HAODA particles (Squalene@HAODA emulsion);
(D) Composition comprising Ocimene@HAODA particles (Ocimene@HAODA emulsion); and
(E) Composition comprising Famesene@HAODA particles (Famesene@HAODA emulsion).

To evaluate the particle size and stability of oil@HAODA particles, aliquots of the compositions comprising oil@HAODA particles were loaded in 1.5-mL Eppendorf tubes and stored separately at 4° C. and 37° C. At predetermined time points, the appearance and particle sizes of the compositions were recorded. The particle size and polydispersity index (PDI) of HAODA and oil@HAODA in the compositions (water solutions) were measured by dynamic light scattering (DLS, Malvern Zetasizer Nano ZS90, Malvem, UK). The HAODA and oil@HAODA were stained by PTA negative staining and their morphology was observed by transmission electron microscopy (TEM, JEOL JEM-1400 electron microscopy, Tokyo, Japan).

Among the different oil@HAODA particles, Paraffin oil@HAODA particles were larger particles, presumably due to fluidity or viscosity characteristic of paraffin oil (an example of mineral oil). In contrast, other terpenoid oil@HAODA particles were stable nanoscale particles suitable for injection. Furthermore, particle size is highly correlated with the efficacy of immune stimulation, and terpenoid oil@HA-derivative particles may have better efficacy as an immune stimulator than mineral oil-forming particles.

### Experiment 2-1-3 the Effects of the Reactants in the Squalene@HAODA Synthesis Process on the Antigen-Presenting Cells (APC) Recruitment

9 BALB/c mice were randomly assigned into 3 groups (three mice per group), which were Hyaluronic acid group, ODA group, and Squalene@HAODA group.

The experimental procedure was roughly the same as that described in Experiment 1-5. The only differences were the compositions administered to the mice.

Hyaluronic acid group: 50 μL of the sodium citrate solution comprising Hyaluronic acid (8.73 mg/mL) (the test sample in this group) was injected intramuscularly into the right quadriceps of mice; and 50 μL of sodium citrate buffer (pH=6.5) (the vehicle control in this group) was injected intramuscularly into the left quadriceps of mice to monitor the effect induced by the injection on each mouse.

ODA group: 50 μL of the mixture solution comprising 0.036 mg of octadecan-1-amine (octadecylamine) dissolved in 25 μL of the N-methyl-2-pyrrolidone (NMP) and 25 μL of the PBS buffer (the test sample in this group) was injected intramuscularly into the right quadriceps of mice; and 50 μL of the mixture solution comprising 25 μL of the N-methyl-2-pyrrolidone (NMP) and 25 μL of the PBS buffer (the vehicle control in this group) was injected intramuscularly into the left quadriceps of mice to monitor the effect induced by the injection on each mouse.

Squalene@HAODA group: 50 μL of the composition comprising Squalene@HAODA particles (octadecan-1-amine content: 0.036 mg; squalene content: 2.5 μL; hyaluronic acid content: 0.4325 mg) prepared by the method as described in Experiment 2-1-2 (the test sample in this group) was injected intramuscularly into the right quadriceps of mice; and 50 μL of sodium citrate buffer (pH=6.5) (the vehicle control in this group) was injected intramuscularly into the left quadriceps of mice in these groups to monitor the effect induced by the injection on each mouse.

The mice in each group were sacrificed at 5^{th} day after injection.

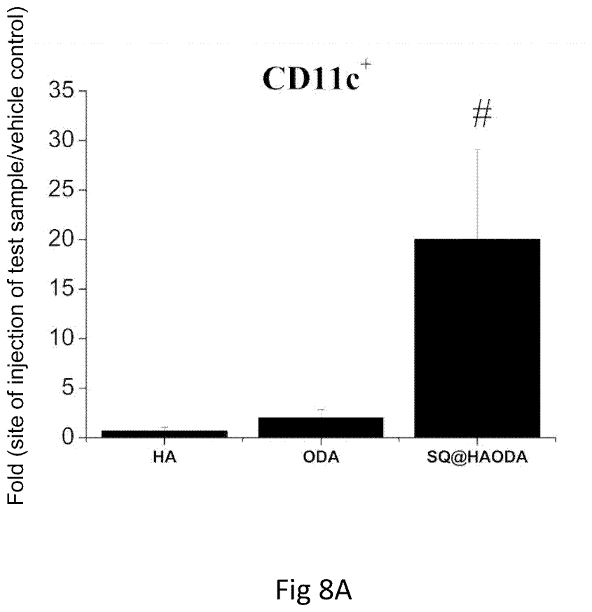

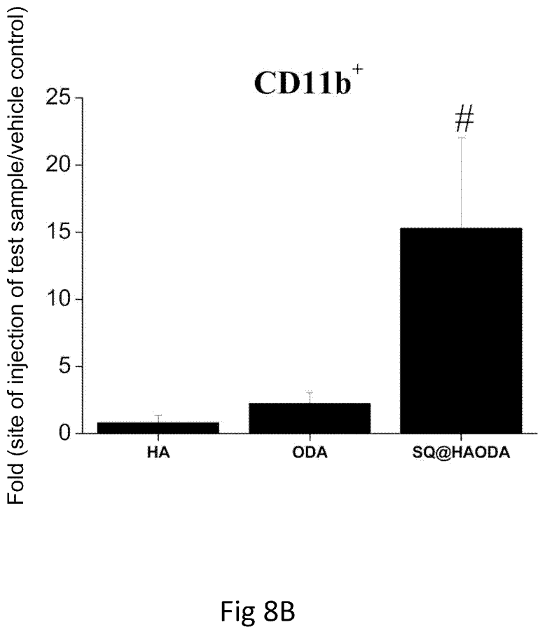

Please refer to FIGS. 8A and 8B. FIG. 8A is result which shows the effect of reactants and product in the Squalene@HAODA synthesis process on dendritic cells recruitment. FIG. 8B is result which shows the effect of reactants and product in the Squalene@HAODA synthesis process on macrophages recruitment. Please note that the symbol “#” means the group is significant difference compared with other three groups

As shown in FIGS. 8A and 8B, all of the reactants and product in the Squalene@HAODA synthesis process (ODA and hyaluronic acid) lack the ability to recruit dendritic cells (CD11c+) and macrophages (CD11b+). Unexpectedly, Squalene@HAODA particles could recruit more than 30 folds of dendritic cells (CD11c⁺) and more than 15 folds of macrophages (CD11b⁺).

As shown in FIG. 8A, comparison among the dendritic cells recruitment efficacy of the ODA group, hyaluronic acid group, and Squalene@HAODA group demonstrated that octadecan-1-amine and hyaluronic acid in the Squalene@HAODA particles manifests synergy in the dendritic cells recruitment efficacy.

As shown in FIG. 8B, comparison among the macrophages recruitment efficacy of the ODA group, hyaluronic acid group, and Squalene@HAODA group demonstrated that octadecan-1-amine and hyaluronic acid in the Squalene@HAODA particles manifests synergy in the macrophages recruitment efficacy.

The foregoing descriptions are merely the preferred embodiments of the present invention and are not intended to limit the scope of the patent application of the present invention. Therefore, any alteration or modification that does not depart from the spirits disclosed herein should be included within the scope of the patent application of the present invention.

All publications mentioned herein are hereby incorporated by reference in their entirety for the purpose of describing and disclosing, for example the compositions and methodologies that are disclosed herein, which might be used in connection with the presently described inventions. The publications discussed herein are provided solely for their disclosure prior to the filing date of the present application. Nothing herein is to be construed as an admission that the inventors described herein are not entitled to antedate such disclosure by virtue of prior invention or for any other reason.

### REFERENCE

1. De Jong, J. C., Palache, A. M., Beyer, W. E., Rimmelzwaan, G. F., Boon, A. C., & Osterhaus, A. D. (2003). Haemagglutination-inhibiting antibody to influenza virus. Developments in biologicals, 115, 63-73.

1. A composition comprising oil and macromolecule; the oil and macromolecule together form a plurality of particles; the macromolecule comprises:
at least one first unit, wherein the first unit is a first disaccharide; and
at least one second unit, wherein the second unit is a conjugation of a second disaccharide and a hydrophobic compound;
wherein the at least one first unit and the at least one second unit are linked to each other to form the macromolecule.

2. The composition of claim 1, wherein the oil is a terpenoid; or
wherein the first disaccharide is composed of D-glucuronic acid and N-acetylglucosamine; or
wherein the second disaccharide is composed of D-glucuronic acid and N-acetylglucosamine; and in the second unit, the hydrophobic compound is attached to carbon 6 of the D-glucuronic acid.

3. The composition of claim 2, wherein the D-glucuronic acid and N-acetylglucosamine in the first disaccharide are linked by a β-(1,3) bond; or
the N-acetylglucosamine and the D-glucuronic acid in the second disaccharide are linked by a β-(1,3) bond;
or the at least the first unit and the at least the second unit are linked to each other by a β-(1,4) bond to form the macromolecule.

4. The composition of claim 1, wherein the macromolecule is a compound of formula (I): 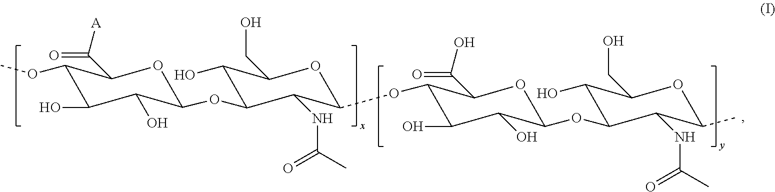
and wherein A group is R—NH— or R—X—NH—; wherein R group is a hydrophobic group derived from the hydrophobic compound, X denotes a hetero-atom (such as O, S, or N), a carbonyl group, or —O—CO—C₁₋₅ alkylene-, wherein the alkylene can be optionally substituted by one or more substituents selected from a group consisting of alkyl, aromatic, anti-aromatic, cycloalkyl, acetyl, amino, hydroxyl, and thiol groups, or an alkane or alkene group, of which the main carbon chain has carbon atom selected from one to ten, such alkane or alkene group can be optionally substituted by one or more substituents selected from a group consisting of alkyl, aromatic, anti-aromatic, cyclic group, carbonyl, acetyl, keto, amino, hydroxyl, and hetero-atom (such as O, S, or N) etc.; or —X—NH— group can be denoted as an amino acid, whose main chain has 2 to 6 carbon atoms, such as glycine, alanine, valine, leucine, isoleucine; x and y each individually represent an integer value, x≥1, y≥x, and x+y≥3; or
the particles are detectable by a particle size analyzer, a transmission electron microscope, or a scanning electron microscope.

5. The composition of claim 1, wherein the composition is an aqueous composition or an oil-in-water composition; or
the macromolecule is an amphiphilic compound and the hydrophilic-lipophilic balance value (HLB value) of the macromolecule is greater than 7.

6. The composition of claim 1, wherein the hydrophobic compound is selected from the group consisting of cholesterol derivatives, octadecylamine, octadecylamine derivatives, steroids, steroid derivatives, saturated or unsaturated long chain fatty amines, and saturated or unsaturated long chain fatty amine derivatives, or any combination thereof.

7. The composition of claim 2, wherein the terpenoid is liquid or solid at room temperature; or
the terpenoid is a cyclic compound or a non-cyclic compound; or
the terpenoid consists of 1˜20 sesquiterpene(s); or
the terpenoid is an unsaturated terpenoid.

8. The composition of claim 1, wherein the oil is selected from the group consisting of squalene, squalane, ocimene, farnesene, and paraffin oil, or any combination thereof.

9. The composition of claim 1, wherein the macromolecule has an average molecular weight of at least 10 kDa and less than or equal to 1000 kDa; or
wherein in the composition, at least 70% by molar ratio of the units per macromolecule on average are the first units.

10. The composition of claim 1, wherein in the composition, the weight ratio of the oil and the macromolecule ranges from 1:100 to 80:1; or
the weight percentages of the oil and macromolecule are respectively equal to or more than 0.01% to 40% and equal to or more than 0.001% to 3%, based on the total weight of the composition; or
at least 50% (w/w) of oil in the composition is encapsulated in the particles, based on the total content of oil in the composition being 100% by mass; or
the composition comprises at least 1% (v/v) of oil, based on the total volume of the composition.

11. The composition of claim 1, wherein the macromolecule is prepared by a method comprising mixing 0.0001˜2 equivalent of the hydrophobic compound with 1 equivalent of hyaluronic acid; or in the composition, the molar ratio of the first unit and the second unit per macromolecule on average ranges from 99:1 to 1:1.

12. A pharmaceutical composition comprising the composition of claim 1.

13. Use of the composition or the pharmaceutical composition of claim 1 in stimulating an immune response in a subject.

14. Use of the composition or the pharmaceutical composition of claim 1 in the treatment or prevention of a disease or disorder.

15. A method of stimulating an immune response in a subject, comprising administering the composition or the pharmaceutical composition of claim 1 to the subject.

16. A method of treating or preventing a disease or disorder in a subject in need thereof, comprising administering the composition or the pharmaceutical composition of claim 1 to the subject.

17. The method of claim 16, wherein the disease or disorder is a tumor or cancer.

18. The method of claim 17, wherein the tumor or cancer is a breast cancer or other solid tumor.

19. The method of claim 16, wherein the disease or disorder is a pathogenic disease, HIV or other viral infection, fungal infection, protozoan infection, or bacterial infection.

20. The use of claim 13, wherein pharmaceutical composition is a vaccine; or the subject is human.
